SEQUENCE LISTING (12 sequences)

SEQ ID NO: 1 - protein, 201 aa, Homo sapiens
  Protein sequence of Human full-length DPT
MDLSLLWVLLPLVTMAWGQYGDYGYPYQQYHDYSDDGWVNLNRQGFSYQCPQGQVIVAVRSIFSKKEGSDRQWNYACMPTPQSLGEPTECWWEEINRAGMEWYQTCSNNGLVAGFQSRYFESVLDREWQFYCCRYSKRCPYSCWLTTEYPGHYGEEMDMISYNYDYYIRGATTTFSAVERDRQWKFIMCRMTEYDCEFANV

SEQ ID NO: 2 - protein, 183 aa, Artificial Sequence
  Protein sequence of Human DPT functional fragment
QYGDYGYPYQQYHDYSDDGWVNLNRQGFSYQCPQGQVIVAVRSIFSKKEGSDRQWNYACMPTPQSLGEPTECWWEEINRAGMEWYQTCSNNGLVAGFQSRYFESVLDREWQFYCCRYSKRCPYSCWLTIEYPGHYGEEMDMISYNYDYYIRGATTTFSAVERDRQWKFIMCRMTEYDCEFANV

SEQ ID NO: 3 - DNA, 606 bp, Homo sapiens
  Nucleotid sequence of Human full-length DPT
atggacctcagtcttctctgggtacttctgcccctagtcaccatggcctggggccagtatggcgattatggatacccataccagcagtatcatgactacagcgatgatgggtgggtgaatttgaaccggcaaggcttcagctaccagtgtccccaggggcaggtgatagtggccgtgaggagcatcttcagcaagaaggaaggttctgacagacaatggaactacgcctgcatgcccacgccacagagcctcggggaacccacggagtgctggtgggaggagatcaacagggctggcatggaatggtaccagacgtgctccaacaatgggctggtggcaggattccagagccgctacttcgagtcagtgctggatcgggagtggcagttttactgttgtcgctacagcaagaggtgcccatattcctgctggctaacaacagaatatccaggtcactatggtgaggaaatggacatgatttcctacaattatgattactatatccgaggagcaacaaccactttctctgcagtggaaagggatcgccagtggaagttcataatgtgccggatgactgaatacgactgtgaatttgcaaatgtttag

SEQ ID NO: 4 - protein, 201 aa, Mus musculus
  mouse DPT (full length)
MDLTLLWVLLPLVTTAWGQYGGYGYPYQQYQDYGDDGWVNLNRQGFSYQCPHGQVVVAVRSIFSKKEGSDRQWNYACMPTPQSLGEPTECWWEEINRAGMEWYQKCSNNGLVAGFQSRYFESVLDREWQFYCCRYSKRCPYSCWMTTEYPSHYGEDMDMISYDYDFYMRGATTTFSAVERDRQWKFIMCRMTDYDCEFENV

SEQ ID NO: 5 - protein, 183 aa, Artificial Sequence
  Recombinant mouse DPT functional fragment
QYGGYGYPYQQYQDYGDDGWVNLNRQGFSYQCPHGQVVVAVRSIFSKKEGSDRQWNYACMPTPQSLGEPTECWWEEINRAGMEWYQKCSNNGLVAGFQSRYFESVLDREWQFYCCRYSKRCPYSCWMTTEYPSHYGEDMDMISYDYDFYMRGATTTFSAVERDRQWKFIMCRMTDYDCEFENV

SEQ ID NO: 6 - DNA, 8835 bp, Artificial Sequence
  Dpt overexpression vector
gtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtccaccaacgagaatgctaatacaccagctgcccgtcttcacagattcaagaacaagggaaaagacagtacagaaatgaggcgtcgcagaatagaggtcaatgtggagctgaggaaagctaagaaggatgaccagatgctgaagaggagaaatgtaagctcatttcctgatgatgctacttctccgctgcaggaaaactaagcggccgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcggccggtcgaatcaagcttatcgataccgtcgacggatccgccaccatggacctcactcttctgtgggttcttctgccactggtgaccacagcctggggccagtatggtggctacgggtacccataccagcagtaccaggactacggtgatgatggatgggtgaatcttaaccgccaaggcttcagctaccagtgtccccacgggcaagtggtggtagctgtgaggagcatctttagcaagaaggaaggctctgacagacaatggaactatgcctgcatgcctacaccccaaagcctgggggagcccactgagtgctggtgggaggagatcaacagggccggcatggaatggtaccagaagtgctccaacaatggattggtggcggggttccagagccgctacttcgaatcagtgctggatcgtgagtggcaattttactgctgccgctatagcaagaggtgtccatattcctgctggatgacaacagagtacccaagccactatggggaagacatggacatgatttcctatgactacgatttttatatgcgaggagcaacaaccactttctctgcagtggaaagggatcgccagtggaagttcatcatgtgccggatgactgactacgactgcgaattcgaaaacgtttagggatccgaattcggccggccatatgtttaaacatgcattaattaactagtctagatttaaataccggtacgtagcggccgcccggggccctccgagcaattcctgcagcccgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccac

SEQ ID NO: 7 - DNA, 58 bp, Artificial Sequence
  shRNA hairpin sequence
ccgggcgaggagcaacaaccactttctcgagaaagtggttgttgctcctcgctttttg

SEQ ID NO: 8 - DNA, 52 bp, Artificial Sequence
  Dpt forward PCR primer
cacggatccgccaccatggacctcactcttctgtgggttcttctgccactgg

SEQ ID NO: 9 - DNA, 34 bp, Artificial Sequence
  Dpt reverse PCR primer
cacggatccctaaacgttttcgaattcgcagtcg

SEQ ID NO: 10 - DNA, 606 bp, Mus musculus
  Nucleotid sequence of mouse full-length DPT (without 3′ and 5′ UTR)
atggacctcactcttctgtgggttcttctgccactggtgaccacagcctggggccagtatggtggctacgggtacccataccagcagtaccaggactacggtgatgatggatgggtgaatcttaaccgccaaggcttcagctaccagtgtccccacgggcaagtggtggtagctgtgaggagcatctttagcaagaaggaaggctctgacagacaatggaactatgcctgcatgcctacaccccaaagcctgggggagcccactgagtgctggtgggaggagatcaacagggccggcatggaatggtaccagaagtgctccaacaatggattggtggcggggttccagagccgctacttcgaatcagtgctggatcgtgagtggcaattttactgctgccgctatagcaagaggtgtccatattcctgctggatgacaacagagtacccaagccactatggggaagacatggacatgatttcctatgactacgatttttatatgcgaggagcaacaaccactttctctgcagtggaaagggatcgccagtggaagttcatcatgtgccggatgactgactacgactgcgaattcgaaaacgtttag

SEQ ID NO: 11 - DNA, 552 bp, Artificial Sequence
  Nucleic acid sequence of Human DPT functional fragment
cagtatggcgattatggatacccataccagcagtatcatgactacagcgatgatgggtgggtgaatttgaaccggcaaggcttcagctaccagtgtccccaggggcaggtgatagtggccgtgaggagcatcttcagcaagaaggaaggttctgacagacaatggaactacgcctgcatgcccacgccacagagcctcggggaacccacggagtgctggtgggaggagatcaacagggctggcatggaatggtaccagacgtgctccaacaatgggctggtggcaggattccagagccgctacttcgagtcagtgctggatcgggagtggcagttttactgttgtcgctacagcaagaggtgcccatattcctgctggctaacaacagaatatccaggtcactatggtgaggaaatggacatgatttcctacaattatgattactatatccgaggagcaacaaccactttctctgcagtggaaagggatcgccagtggaagttcataatgtgccggatgactgaatacgactgtgaatttgcaaatgtttag

SEQ ID NO: 12 - DNA, 552 bp, Artificial Sequence
  Nucleic acid sequence of mouse DPT functional fragment
cagtatggtggctacgggtacccataccagcagtaccaggactacggtgatgatggatgggtgaatcttaaccgccaaggcttcagctaccagtgtccccacgggcaagtggtggtagctgtgaggagcatctttagcaagaaggaaggctctgacagacaatggaactatgcctgcatgcctacaccccaaagcctgggggagcccactgagtgctggtgggaggagatcaacagggccggcatggaatggtaccagaagtgctccaacaatggattggtggcggggttccagagccgctacttcgaatcagtgctggatcgtgagtggcaattttactgctgccgctatagcaagaggtgtccatattcctgctggatgacaacagagtacccaagccactatggggaagacatggacatgatttcctatgactacgatttttatatgcgaggagcaacaaccactttctctgcagtggaaagggatcgccagtggaagttcatcatgtgccggatgactgactacgactgcgaattcgaaaacgtttag

(19) United States
(12) Patent Application Publication
(10) Pub. No.: US 2018/0230433 A1
(43) Pub. Date: Aug. 16, 2018

(54) USE OF DERMATOPONTIN FOR MAINTAINING HEMATOPOIETIC STEM AND/OR PROGENITOR CELLS IN CULTURE

(71) Applicants: HELMHOLTZ ZENTRUM MÜNCHEN - DEUTSCHES FORSCHUNGSZENTRUM FÜR GESUNDHEIT UND UMWELT (GMBH), Neuherberg (DE); EIDGENÖSSISCHE TECHNISCHE HOCHSCHULE ZÜRICH, Zürich (CH)

(72) Inventors: Konstantinos KOKKALIARIS, Basel (CH); Timm SCHROEDER, Basel (CH)

(21) Appl. No.: 15/750,132

(22) PCT Filed: Aug. 5, 2016
(86) PCT No.: PCT/EP2016/068790

§ 371 Date: Feb. 2, 2018

(30) Foreign Application Priority Data
Aug. 5, 2015 (EP) 15179829.5
May 13, 2016 (EP) 16169597.8

(51) Int. Cl. C12N 5/0789

(52) U.S. Cl. CPC C12N 5/0647; C12N 2500/90; C12N 2501/998; C12N 2502/13; C12N 2501/145; C12N 2533/90; C12N 2533/54; C12N 2501/125

(57) ABSTRACT

The present invention relates to the use of dermatopontin (DPT) or a functional fragment thereof for the maintenance of hematopoietic stem and/or progenitor cells in culture. The present invention further relates to a method for maintaining hematopoietic stem and/or progenitor cells in culture, the method comprising culturing the hematopoietic stem and/or progenitor cells in the presence of dermatopontin (DPT) or a functional fragment thereof. Furthermore, the present invention relates to a cell culture medium for the maintenance of hematopoietic stem and/or progenitor cells, wherein the cell culture medium comprises a medium and dermatopontin (DPT) or a functional fragment thereof and further optionally comprises serum/serum replacement, (a) reducing agent(s), and/or (an) antibiotic(s) as well as a kit comprising dermatopontin (DPT) or a functional fragment thereof and at least one of: (a) (a) cell culture medium; (b) one or more cytokines; (c) serum/serum replacement; (d) (a) reducing agent(s), and/or (e) (an) antibiotic(s).

The present invention relates to the use of dermatopontin (DPT) or a functional fragment thereof for the maintenance of hematopoietic stem and/or progenitor cells in culture. The present invention further relates to a method for maintaining hematopoietic stem and/or progenitor cells in culture, the method comprising culturing the hematopoietic stem and/or progenitor cells in the presence of dermatopontin (DPT) or a functional fragment thereof. Furthermore, the present invention relates to a cell culture medium for the maintenance of hematopoietic stem and/or progenitor cells, wherein the cell culture medium comprises a medium and dermatopontin (DPT) or a functional fragment thereof and further optionally comprises serum/serum replacement, (a) reducing agent(s), and/or (an) antibiotic(s) as well as a kit comprising dermatopontin (DPT) or a functional fragment thereof and at least one of: (a) (a) cell culture medium; (b) one or more cytokines; (c) serum/serum replacement; (d) (a) reducing agent(s), and/or (e) (an) antibiotic(s).

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Stem cells have the potential to generate, regenerate and repair tissues by producing large numbers of tissue-specific differentiated cell types life-long. Hematopoietic stem cells (HSCs) have the capacity to daily produce blood cells of all lineages, while—in vivo—maintaining their undifferentiated state long-term, even after numerous cell divisions. HSCs can also replenish the blood system of recipient organisms upon transplantation; therefore they can be used in regenerative medicine against blood disorders, injuries and for hematopoietic recovery after irradiation/chemotherapy treatment of various cancers. This holds true for HSCs with and without genetic manipulation.

The clinical application of HSCs is hampered by the current inability to maintain these cells ex vivo. Despite numerous attempts, only a limited number of in vitro systems capable of maintaining or expanding HSCs have been reported so far (Sorrentino, 2004). These systems can be divided into those that focus on the manipulation of intrinsic factors, e.g. transcription factors, and those that rely on extrinsic factors, e.g. cytokines or stromal cell co-cultures.

One approach to maintain/expand HSCs is based on the retroviral over-expression of the homeodomain transcription factor HOXB4. Antonchuk et al., 2002 reported a 40-fold expansion of transduced HSC in vitro, without leading to hematopoietic malignancies upon serial transplantation into murine recipients (Sauvageau et al., 1995). Due to unpredicted effects of transcription-factor overexpression on human recipients and the reported oncogenic potential of viral gene transfer systems (Baum, 2004), a viral-free system was later developed. In this system, the HOXB4 protein was fused to a sequence allowing plasma-membrane permeabilisation. However, technical problems hampered the further application of this approach and no successful application for therapeutic expansion of HSCs has been reported so far.

An alternative method currently used to maintain/expand HSCs is via the addition of specific molecules, such as hematopoietic cytokines. These cytokines are small signaling molecules controlling the function and the behaviour of hematopoietic cells. Over the last decades, a plethora of single cytokines and their combinations have been extensively studied, including the stem cell factor (SCF), thrombopoietin (TPO), angiopoietin 1 (Ang1), granulocyte colony-stimulating factor (G-CSF), Flt3-ligand (Flt3L), pleiotrophin (Ptn), interleukin 3 (IL-3), 6 (IL-6) and 11 (IL-11). Early studies reported that culturing HSCs in SCF, IL3 and IL6 promoted their self-renewal (Bodine et al., 1989, 1992). However, later studies supported that SCF and TPO were more potent for HSC self-renewal in culture (Ema et al., 2000; Takano et al., 2004), while other studies challenged the positive role of TPO(Sekulovic et al., 2011). The fact that each cytokine can have multiple roles in HSC behaviour (Metcalf, 2008), often depending on its concentration, further complicates their use. At present, the most promising conditions for HSC expansion include the combination of cytokines (SCF, TPO) and growth factors, for example insulin-like growth factor 2 (IGF2), fibroblast growth factor (FGF1) and angiopoietin-like proteins, such as Angptl2 or Angtpl3 (Zhang et al., 2006). However, the reported experiments contained a very limited number of HSCs, with later studies reporting loss of HSCs after such treatment (Wohrer et al., 2014). The use of cytokine combinations in human HSC samples also had only limited success leading to 2-4 fold increase over input cells (Takizawa et al., 2011) and the reported effects were only observed during short culture periods, since periods longer than three days resulted in considerable loss of HSCs (Ema et al., 2000; Noda et al., 2008).

A further approach currently under investigation is based on extracellular matrix (ECM) proteins. In the in vivo niche, ECM proteins facilitate the interaction between HSCs and cytokines or cell adhesion molecules, thus regulating HSC fates by providing a 3D scaffold. As an example, fibronectin fibers immobilize soluble factors (i.e. osteopontin), thereby enabling their binding to CD44 or integrins expressed by HSCs (Wilson and Trumpp, 2006). Alternatively, ECM proteins can directly bind to HSC receptors, like hyaluronic acid binding to CD44. However, so far, several attempts to recapitulate the in vivo ECM environment had limited success when applied to ex vivo HSC culture, mainly due to technical limitations (Aggarwal et al., 2012; Lane et al., 2014; Prewitz et al., 2013). One approach to circumvent those limitations is by de-cellularization of cell culture-derived matrices. Previous studies showed that such methods support culture of mesenchymal stem cells or human HSCs in vitro (Chen et al., 2007; Prewitz et al., 2013). However, in these ill-defined conditions, the exact molecular players responsible for the observed stem-cell support remain obscure. This is problematic as regulatory approval for any kind of clinical use typically requires well defined conditions, including the knowledge of the molecules involved. Furthermore, optimisation of such conditions is hampered by the lack of knowledge of the participating molecules.

Studies that focused on a number of ECM candidates were so far only carried out for cultures of human CD34+ hematopoietic progenitor cells, that were supplemented with cytokine cocktails in stroma-free cultures, such as fibronectin (Feng et al., 2006), collagen 1 (Oswald et al., 2006) or heparin sulfate (Punzel et al., 2002). As the human CD34+ hematopoietic progenitor cell fraction only contains negligible amounts of functional HSCs (<0.5%), these studies do not allow any conclusions about the possible effects of these molecules on HSCs, in particular as it has been shown that whole bone marrow or such un-purified or only minimally purified populations behave differently from hematopoietic stem and/or progenitor cells (Challen et al., 2009; Wilson et al., 2008)

Thus, with the presently available stroma-free culture conditions, HSCs cannot robustly be maintained or expanded longer than a few days.

Accordingly, the most efficient method to date is based on mimicking the interaction between HSCs and niche cells. Very few niche cell lines have been reported to support ex vivo HSC maintenance for long culture periods. These cells include e.g. AFT024 (Moore et al., 1997a; Nolta et al., 2002), EL08 (Oostendorp et al., 2002) and UG26-1B6 stroma cell lines (Oostendorp et al., 2002). Among those, only the clonal, fetal liver-derived cell line AFT024 has been reported to qualitatively and quantitatively maintain murine (Moore et al., 1997) and human HSCs (Nolta et al., 2002) for extended culture periods of up to several weeks.

Thus, despite the fact that a lot of effort has been invested into screening a large number of molecules, current culture methods are inadequate to robustly maintain or expand HSCs ex vivo long-term, without changing the properties of these cells, i.e. without the need for artificial modifications of the cells such as cell transformation with viral oncogenes or rendering the cells tumorigenic. Accordingly, there is still a need to provide such methods and such cell cultures.

This need is addressed by the provision of the embodiments characterised in the claims.

Accordingly, the present invention relates to the use of dermatopontin (DPT) or a functional fragment thereof for the maintenance of hematopoietic stem and/or progenitor cells in culture.

Dermatopontin (abbreviated herein as DPT (for the protein) or Dpt or Dpt (for the nucleic acid) is an extracellular matrix protein that is considered to mediate adhesion by cell surface integrin binding (Forbes et al., 1994; Superti-Furga et al., 1993). Further, it is believed that DPT serves as a communication link between the dermal fibroblast cell surface and its extracellular matrix environment, thus functioning in cell-matrix interactions and matrix assembly. Human DPT is for example represented by the Uniprot accession number Q07507 or NCBI accession number AAH33736.1 (as of Sep. 23, 2014) and is shown in SEQ ID NO:1. Human DPT has been described in the art, e.g. in Superti-Furga et al., 1993 or Vanderperre et al., 2013. Murine DPT is for example represented by the Uniprot accession number Q9QZZ6 and is shown in SEQ ID NO:4. The terms “dermatopontin” or “DPT”, as used herein, refer to the protein, unless otherwise specified.

The term “functional fragment” relates to a DPT sequence that is shorter than full-length but that maintains or essentially maintains the biological function of DPT. It is well known in the art that functional polypeptides may be shortened to yield fragments with unaltered or substantially unaltered function. In accordance with the present invention, DPT protein activity is essentially retained, if at least 60% of the biological activity of DPT are retained. Preferably, at least 75% or at least 80% of DPT protein activity are retained. More preferred is that at least 90% such as at least 95%, even more preferred at least 98% such as at least 99% of the biological activity of DPT are retained. Most preferred is that the biological activity is fully, i.e. to 100%, retained. Also in accordance with the invention are functional fragments having increased biological activity compared to the full-length DPT protein, i.e. more than 100% activity. Methods of assessing the biological activity of DPT are well known to the person skilled in the art and include, without being limiting, its ability to modulate collagen fibrillogenesis (Takeda et al., 2002). In addition, methods of assessing the biological activity of DPT include the effect of DPT on the maintenance of hematopoietic stem and/or progenitor cells in culture, as shown in the appended examples. Thus, comparing the effect of a fragment of interest and a full-length DPT on the maintenance of hematopoietic stem and/or progenitor cells in culture provides one approach to determine whether the biological function of DPT has been maintained in the fragment.

Such fragments of DPT include, for example, fragments wherein a given number of N- and/or C-terminal amino acids have been removed. Additionally or alternatively, a number of internal (non-terminal) amino acids may be removed, provided the obtained fragment maintains the function of DPT. Said number of amino acids to be removed from the termini and/or internal regions may be one, two, three, four, five, six, seven, eight, nine, ten, 15, 20, 25, 30, 40, 50 or more than 50. Any other number between one and 50 is also deliberately envisaged. Preferably, the fragment retains a length of at least 35 amino acids, more preferably between 150 and 190 amino acids and most preferably between 170 and 185 amino acids.

Preferably, the removal of amino acids is carried out such that the sequence and boundaries of conserved functional domain(s) or sub-sequences in the sequence of DPT is not affected. Means and methods for determining such domains are well known in the art and include experimental and bioinformatic means. Experimental means include the systematic generation of deletion mutants and their assessment in assays for DPT activity known in the art and as described in the examples enclosed herewith. Bioinformatic means include database searches. Suitable databases included protein sequence databases. In this case a multiple sequence alignment of significant hits is indicative of domain boundaries, wherein the domain(s) is/are comprised of the/those subsequences exhibiting an elevated level of sequence conservation as compared to the remainder of the sequence. Further suitable databases include databases of statistical models of conserved protein domains such as Pfam maintained by the Sanger Institute, UK (see the world wide web at sanger.ac.uk/Software/Pfam).

So far, two main domains of DPT have been identified, namely the N-terminal “signal peptide domain” ranging from amino acids 1 to 18 and the “polypeptide chain in the mature protein following processing” ranging from amino acids 19 to 201. Accordingly, a preferred functional fragment in accordance with the present invention is a fragment ranging from amino acid 19 to 201 of the amino acids shown in SEQ ID NO:1. The sequence of such a preferred functional fragment of human DPT is shown in SEQ ID NO: 2 and in SEQ ID NO: 5 for a functional fragment of murine DPT.

The DPT or functional fragment thereof may further be fusion proteins, wherein the fusion partner is attached N- or C-terminally to the DPT or functional fragment thereof. The fusion partner, i.e. the components of said fusion proteins that are not DPT sequences or fragments thereof as defined herein above, include amino acid sequences which confer desired properties such as modified/enhanced stability, modified/enhanced solubility and/or simplified purification of expressed recombinant product. Non-limiting examples of such fusion partners include a His-tag, a Strep-tag, a GST-tag, a TAP tag, biotin, an HA tag or a signal sequence for extracellular targeting. Signal sequences for extracellular targeting include, without being limiting, secretion signals. Preferably, the fusion partner is a C-terminal His-tag.

DPT (or a functional fragment thereof) can be obtained commercially, for example from R&D systems. Alternatively, DPT or a functional DPT fragment can be expressed recombinantly by methods known in the art, e.g. by expressing a vector encoding DPT or the fragment thereof in a suitable host and purifying the expressed DPT.

A large number of suitable methods exist in the art to produce proteins in appropriate hosts. If the host is a unicellular organism such as a prokaryote, a mammalian or insect cell, the person skilled in the art can revert to a variety of culture conditions. Conveniently, the produced protein is harvested from the culture medium, lysates of the cultured organisms or from isolated (biological) membranes by established techniques. In the case of a multi-cellular organism, the host may be a cell which is part of or derived from a part of the organism, for example said host cell may be the harvestable part of a plant. A preferred method involves the recombinant production of protein in hosts as indicated above. For example, nucleic acid sequences comprising the polynucleotide according to the invention can be synthesized by PCR and inserted into an expression vector. Subsequently a suitable host may be transformed with the expression vector. Thereafter, the host is cultured to produce the desired polypeptide(s), which is/are isolated and purified.

An alternative method for producing the polypeptide of the invention is in vitro translation of mRNA. Suitable cell-free expression systems for use in accordance with the present invention include rabbit reticulocyte lysate, wheat germ extract, canine pancreatic microsomal membranes, E. coli S30 extract, and coupled transcription/translation systems such as the TNT-system (Promega). These systems allow the expression of recombinant polypeptides upon the addition of cloning vectors, DNA fragments, or RNA sequences containing coding regions and appropriate promoter elements.

In addition to recombinant production, the protein or fragments of the invention may be produced synthetically, e.g. by direct peptide synthesis using solid-phase techniques (cf Stewart et al. (1969) Solid Phase Peptide Synthesis; Freeman Co, San Francisco; Merrifield, J. Am. Chem. Soc. 85 (1963), 2149-2154). Synthetic protein synthesis may be performed using manual techniques or by automation. Automated synthesis may be achieved, for example, using the Applied Biosystems 431A Peptide Synthesizer (Perkin Elmer, Foster City Calif.) in accordance with the instructions provided by the manufacturer. Various fragments may be chemically synthesized separately and combined using chemical methods to produce the full length molecule. As indicated above, chemical synthesis, such as the solid phase procedure described by Houghton Proc. Natl. Acad. Sci. USA (82) (1985), 5131-5135, can be used. Furthermore, the protein or fragments of the protein of the invention may be produced semi-synthetically, for example by a combination of recombinant and synthetic production.

Protein isolation and purification can be achieved by any one of several known techniques; for example and without limitation, ion exchange chromatography, gel filtration chromatography and affinity chromatography, high pressure liquid chromatography (HPLC), reversed phase HPLC, and preparative disc gel electrophoresis. Protein isolation/purification techniques may require modification of the proteins of the present invention using conventional methods. For example, a histidine tag can be added to the protein to allow purification on a nickel column. Other modifications may cause higher or lower activity, permit higher levels of protein production, or simplify purification of the protein.

The amounts of DPT or a functional DPT fragment to be used can be determined by the skilled person without further ado, for example by culturing hematopoietic stem and/or progenitor cells in the presence of varying amounts of DPT or a functional DPT fragment and assessing its effect on cell maintenance. Preferred amounts are detailed further below.

“Hematopoietic stem cells” are well known in the art and are abbreviated as “HSC”, “HSCs” or “HSC(s)” herein. All differentiated blood cells from the lymphoid and myeloid lineages arise from HSCs, which are multipotent, self-renewing cells. Since mature blood cells are predominantly short lived, HSCs continuously provide more differentiated progenitors characterised by a progressive loss of differentiation potential while maintaining the HSC pool size by balancing self-renewal and differentiation. During differentiation, HSC initially produce multipotent progenitors (abbreviated as ‘MPPs’) which then further differentiated to progenitors with restricted lineage potential. Importantly, MPPs still have the potential to produce cells of all lineages, but have reduced self-renewing potential. During the last decades, major efforts have been made towards the identification of markers for the prospective HSC isolation (Osawa et al., 1996, Kiel et al., 2005, Kent et al., 2009). Current marker combination, such as CD150, CD48 (Kiel et al., 2005), CD34 (Osawa et al., 1996), cKit (Okada et al., 1991), Sca-1 (Spangrude et al., 1988) and EPCR (Kent et al., 2009) allow sorting HSCs with purities of approximately 50%.

“Hematopoietic progenitor cells” are also well known in the art and are also referred to herein as multipotent progenitors (MPPs). MPPs have a less robust self-renewal capacity than HSCs, but they are still multipotent, i.e. they can give rise to all types of mature blood cells (Morrison et al., 1997; Weissman, 2000). MPPs are of particular clinical relevance, as they are the cells responsible for the short-term repopulation/regeneration of the blood system (up to 16 weeks), until HSCs can start fulfilling this role.

It will be appreciated that hematopoietic stem cells and hematopoietic progenitor cells are committed to differentiate into hematopoietic cells and, consequently, are more differentiated than embryonic stem cells.

Hematopoietic stem and/or progenitor cells can be obtained from a variety of donors, such as e.g. mammalians as well as non-mammalian animals, such as Danio rerio, Drosophila melanogaster, Xenopus laevis, or Gallus (Chicken). Suitable sources for hematopoietic stem and/or progenitor cells include, without being limiting, bone marrow, peripheral blood, umbilical cord blood, the non-human fetal hematopoietic system as well as non-human embryonic stem cells and embryonic germ cells. Such sources, as well as means and methods of obtaining hematopoietic stem and/or progenitor cells from theses sources, are well known in the art and have been described e.g. in Ma et al., 2011 or Notta et al., 2011 as well as in example 1 below.

The term “maintenance”, as used herein, relates to the ability of hematopoietic stem cells and/or hematopoietic progenitor cells to be cultured without differentiating into more mature cell types. During this non-differentiating cell culture, the hematopoietic stem cells and/or hematopoietic progenitor cells can, but do not have to, divide. Accordingly, as used herein, the term “maintenance” encompassed both the preservation of the cell number of hematopoietic stem cells and/or hematopoietic progenitor cells in culture, as well as the expansion of the cell number of hematopoietic stem cells and/or hematopoietic progenitor cells in culture. Preferably, the cells maintain their ability to stay in culture without differentiation for at least 1 week, such as e.g. at least 2 weeks, more preferably at least 3 weeks and even more preferably at least 4 weeks, such as e.g. at least 5 weeks. Most preferably, the cells maintain their ability to stay in culture without differentiation for an unlimited amount of time.

In those cases where the hematopoietic stem cells and/or hematopoietic progenitor cells divide in culture, it is preferred that the rate of cell division (i.e. proliferation) of these cells does not or does not substantially decrease upon continued culture as defined above. Accordingly, the rate of proliferation after the above defined preferred durations of culture, such as e.g. 5 weeks of culture, remains substantially the same or even increases (i.e. the cell number expands) as compared to the rate of proliferation observed directly after the hematopoietic stem cells and/or hematopoietic progenitor cells have been placed in culture (i.e. the initial rate of proliferation). The rate of proliferation is considered to have not substantially decreased as compared to the initial rate of proliferation if it is at least 70% of the initial rate of proliferation, such as e.g. at least 80% of the initial rate of proliferation, more preferably at least 90% of the initial rate of proliferation, such as e.g. at least 95% of the initial rate of proliferation. More preferably, the rate of proliferation is identical to the initial rate of proliferation, but the rate of proliferation can also be higher than the initial rate of proliferation. Such an expansion of cell numbers is explicitly envisaged in accordance with the present invention. The skilled person is well aware of how to determine the rate of proliferation. Non-limiting example include determination of the frequency of splitting of the cells required or of cell numbers after a certain period of growth, such as e.g. 24 hours after splitting etc. (Pollard J W., Basic cell culture. Methods Mol Biol. 1990; 5:1-12.).

In accordance with the present invention, the cells do not differentiate into more mature cell types during the cell culture, i.e. the cells maintain or essentially maintain the characteristics of hematopoietic stem and/or progenitor cells during culture, even after prolonged periods of time. Such characteristics include, without being limiting, their biological function, including without limitation their capability to differentiate into more mature cell types under different conditions, or their specific marker expression profile.

One example of the biological function of hematopoietic stem and/or progenitor cells is their capacity to differentiate under appropriate conditions into all types of mature blood cells, i.e. into red blood cells, platelets, monocytes/macrophages, eosinophils, basophils and neutrophils. Further non-limiting examples of the biological functions of hematopoietic stem and/or progenitor cells include the long-term regeneration or repair of host hematopoiesis upon their transplantation into a recipient host. In addition, their specific marker expression profile includes the presence of e.g. CD150, Sca1, c-Kit, Eper, CD105, CD49b, and/or Thy1.1 and/or a lack or low expression of CD34, CD48, CD3e, CD4, CD8, CD19, B220, TER119, Gr-1, Mac-1, CD244, and/or CD135. This marker set is for example specific for hematopoietic stem and/or progenitor cells in mice. Preferably, the marker expression profile of human HSCs includes expression of CD34, Thy1, c-Kit and lack or low expression of CD38, CD45RA, CD4, CD8, CD19, B220, TER119, Gr-1, and Mac-1.

Means and methods for determining these characteristics are well established in the art. For example, the multipotency of HSCs or MPPs may be determined via methods well known in the art, such as e.g. quantification of the myeloid and lymphoid lineage output of donor-derived cells after transplantation into recipient mice (Osawa et al., 1996), or in vitro colony assays as described in e.g. Muller-Sieburg et al., 2004. The self-renewal potential of HSCs is determined by their potential to reconstitute secondary recipients, as described in Muller-Sieburg et al., 2004.

Moreover, the expression of specific markers can be determined on the amino acid level as well as on the nucleic acid level by methods well known in the art.

In accordance with the present invention, the hematopoietic stem and/or progenitor cells are considered to essentially maintain their characteristics during cell culture if the degree of similarity between the cells at the beginning of the culture and the cells after maintenance in culture, such as e.g. after 1 week, is at least 70%, more preferably at least 80%, such as e.g. at least 90% and more preferably at least 95%. Even more preferably, the degree of similarity is at least 99%, most preferably 100%. The degree of similarity can be determined based on their differentiation potential before and after culture as can be compared and quantified through in vitro colony assays or analysis of the lineage output of transplanted cells, as mentioned above. In addition, their potential before and after culture to reconstitute hematopoiesis of a recipient mouse can be compared and quantified as mentioned above. In addition, the expression profile of the cells after passaging may be compared to the expression profile of cells at the beginning of the culture and the degree of similarity may be determined.

In accordance with the invention, the cell culture is carried out under suitable cell culture conditions. General cell culture conditions as well as suitable cell culture media are well known in the art (e.g. Cooper G M (2000). “Tools of Cell Biology”, ISBN 0-87893-106-6; K. Turksen, ed., Humana Press, 2004, J. Masters, ed., Oxford University Press, 2000, “Animal cell culture”, ISBN-10 0-19-963796-2). Preferably, the cell culture conditions comprise conditions of about 2 to 10% CO₂, preferably about 5% CO₂ and a temperature of about 32 to 38° C., preferably about 37° C. Preferably, the cell culture is carried out under sterile conditions. The cells may be cultured for e.g. about one day, two days, three days, four days, five days, six days, seven days, eight days, nine days, ten days, eleven days, twelve days, 13 days, 14 days, 15 days, 16, days, 17 days, 18 days, 19 days, 20 days or most preferably for about 21 days in accordance with the method of the invention.

Any medium suitable as a culture medium for multipotent term or progenitor cells may be employed for the cell culture. Such media are well established in the art. For example, the culture medium can be a medium selected from the group consisting of DMEM, RPMI 1640, Iscove's, F12, OPTI-MEM, etc. Preferably, the cell culture medium is a basal cell culture medium comprising: (i) serum or serum replacement; (ii) a reducing agent, such as e.g. β-mercaptoethanol; and (iii) (an) antibiotic(s), such as e.g. penicillin G, streptomycin sulfate, Anti-PPLO agent tylosin, Amphotericin B, gentamicin sulfate, kanamycin sulfate, neomycin sulfate, nystatin, polymixin B sulfate, carbenicillin, cefotaxime, chloramphenicol, G418 disulfate salt, hygromycin B, paromycin, rifampicin, and/or vancomycin. Even more preferably, the cell culture medium is DMEM, preferably high glucose DMEM, comprising: (i) serum; (ii) β-mercaptoethanol as the reducing agent; and (iii) penicillin/streptomycin as antibiotics.

DMEM is well known in the art and refers to Dulbecco's Modified Eagle Medium. High glucose DMEM is a basal medium for supporting the growth of many mammalian cells, including multipotent stem and progenitor cells. DMEM can be commercially obtained, for example from Gibco (Cat no.: 11960-085). Also serum or serum replacement can be obtained from Gibco, as well as from PAA. For example fetal calf serum can be obtained from PAA under catalogue number A15-101 and horse serum can be obtained from Gibco under catalogue number 16050-122. β-mercaptoethanol can be commercially obtained, for example from Sigma (Cat. no.: M3142-25ML), while penicillin and streptomycin can for example be obtained from Gibco (Cat. no.: 15140-122).

In an alternative preferred embodiment, the cell culture medium is a serum-free medium, such as e.g. StemSpan SFEM, (StemCell Technologies, Cat no: 09650DMEM). Preferably, said cell culture medium comprises: (i) stem cell factor (SCF, Peprotech, Cat no: 250-03), for example at a concentration of 100 ng/ml; and (ii) thrombopoietin (Tpo, Peprotech, Cat no: 3150-14), for example at a concentration of 100 ng/ml. Such a medium is particularly suitable for the culture of hematopoietic stem and/or progenitor cells in the absence of stroma cells. Additional stroma-free conditions for in vitro culture of hematopoietic stem and/or progenitor cells may include, without being limiting: scove's medium (STEMCELL Technologies) supplemented with BSA (e.g. 10 mg/ml), insulin (e.g. 10 mg/ml), transferrin (e.g. 200 mg/ml), low-density lipoproteins (e.g. 40 mg/ml), penicillin (e.g. 100 U/ml) and streptomycin (e.g. 100 mg/ml); or Iscove's modified Dulbecco's media (IMDM, Thermo Fisher Scientific) supplemented with a-thioglycerol (e.g. 7.5×10^{-5M}), FBS (e.g. 4%), BSA (e.g. 0.1%), transferrin (e.g. 5 mg/ml) and insulin (e.g. 5 mg/ml) or Stem-Pro-34 SFM (Life Technologies) supplemented with β-mercaptoethanol (e.g. 5×10⁻⁵M) and L-glutamine (e.g. 2 mM).

The skilled person is aware of suitable amounts of these compounds to be employed in a cell culture medium for use in the present invention. Preferably, the serum is added as a combination of fetal calf serum and horse serum in an amount of at least 5% each, more preferably at least 7.5% each, and most preferably at least 10% each. If employing serum replacement, the same amounts apply. Preferably, the reducing agent (such as β-mercaptoethanol) is added in an amount of at least 0.01 mM, such as e.g. at least 0.02 mM, at least 0.03 mM, at least 0.04 mM and most preferably at least 0.05 mM. Penicillin and streptomycin mixtures typically consist of 10,000 Units/ml penicillin and 10 mg/ml streptomycin and are preferably added to the cell culture medium in an amount of at least 0.05%, more preferably at least 0.1%.

In a preferred embodiment, the cell culture medium is high glucose DMEM comprising 10% fetal calf serum, 10% horse serum, 0.05 mM β-mercaptoethanol and 0.1% penicillin/streptomycin. This medium is preferred for culture with feeder cells. In another preferred embodiment, the cell culture medium is serum-free StemSpan SFEM comprising 100 ng/ml stem cell factor (SCF) and 100 ng/ml thrombopoietin. This medium is preferred for culture without feeder cells.

In accordance with the present invention, it is envisaged that the cell culture conditions can further comprise the presence of additional cells other than the hematopoietic stem and progenitor cells to be maintained. Such cells can be any cells that are known in the art to be beneficial for the maintenance of hematopoietic stem and progenitor cells, such as for example feeder cells.

The term “feeder cells”, as used herein, is well known in the art and refers to cells that are typically grown to form a coating and supportive layer on cell culture dishes, on which cells can grow which cannot grow on culture dishes devoid of such feeder cells, such as hematopoietic stem and/or progenitor cells. The feeder cells not only provide the physical contact that these cells require for survival and expansion, but also secrete mostly unknown cytokines in the medium. Usually, feeder cells are adherent, growth-arrested but viable and bioactive cells that typically have been irradiated. Stromal cells are an example of commonly employed feeder cells. The term “stromal cells” refers to a collection of different supporting cell types found in tissues or organs and are distinguished from the functional elements of these tissues or organs, i.e. the parenchymal cells. In accordance with the present invention, feeder cells may be obtained from established cell cultures. Alternatively, feeder cells can be autologous, patient-derived feeder cells, i.e. cells obtained from the same patient from which the HSCs or progenitor cells are derived that are to be cultured in accordance with the present invention.

Preferably, such feeder cells do not endogenously express sufficient amounts of DPT or a functional fragment thereof to maintain hematopoietic stem and/or progenitor cells in culture. It is even more preferred that such feeder cells do not endogenously express DPT or a functional fragment thereof. The term “endogenously express” refers to the naturally occurring expression of DPT or a functional fragment thereof in feeder cells. Whether or not a feeder cell line expresses sufficient amounts of DPT or a functional fragment thereof to maintain hematopoietic stem and/or progenitor cells in culture can be tested by the skilled person without further ado, for example, by co-culturing these feeder cells with hematopoietic stem and/or progenitor cells and analysing their efficiency in maintaining these cells in culture. If an effect on the maintenance of the cells is observed, it can be analysed by e.g. targeted knock-out of DPT in the feeder cells, as shown in the appended examples, whether the effect is indeed mediated by this protein or by other factors. Non-limiting examples of such feeder cells that may be used in accordance with the present invention is the stroma cell line 2018 employed in the appended examples.

Most preferably, the cell culture does not contain feeder cells.

In the absence of feeder cells, it is preferred that the surface of the cell culture dish is treated to render it more suitable for cell attachment. Such treatment, also referred to herein as coating, is well known in the art and includes treatment with substances such a e.g. poly-lysine, collagen or other extracellular matrix proteins, phospholipids, antibodies etc. For example, in the absence of feeder cells, plates can be coated with 0.1% gelatin (Sigma Aldrich, Cat no: G1890-100G) for at least one hour at 37° C. Also in the presence of feeder cells, a coating step of the cell culture dish with any of the above described substances can be included, preferably a coating step with 0.1% gelatin for at least one hour at 37° C. In accordance with the present invention, the coating can also be carried out by treatment of the surface of the cell culture dish with DPT, either alone or in combination with established coating substances such as e.g. the coating substances referred to above. Means and methods for the covalent or non-covalent coupling of a protein such as DPT to a cell culture dish are well known in the art (see e.g. Mosiewicz et al., 2013).

As discussed herein above, the influence of feeder cells on the cells in cell culture are complex and not particularly well defined. For therapeutic applications in humans, this can represent an undesired drawback. To avoid this drawback, it is preferred that the cells are cultured in accordance with the present invention and in the absence of feeder cells.

In accordance with the present invention, it was surprisingly found that dermatopontin or a functional fragment thereof enables the robust maintenance of hematopoietic stem and progenitor cells ex vivo for at least 1 week.

As discussed herein above, numerous methods have been described in the art in order to maintain these cells ex vivo. However, these methods either require the presence of additional allogeneic (feeder) cells, the overexpression of specific genes or the presence of hematopoietic cytokines. The methods relying on the overexpression of specific genes, typically transcription factors, are often associated with a risk of oncogenic transformation. Methods making use of cytokines during the cell culture conditions are not associated with this risk, but suffer instead from poor results, mainly due to high cell death rates and undesirably high degree of differentiation of the cells. The suitability of extracellular matrix (ECM) proteins has also been investigated, however not for adequately purified hematopoietic stem and progenitor cells per se. Overall, the presently available feeder cell-free culture conditions are not suitable to maintain HSCs longer than a few days. Whereas the use of feeder cells provides more promising results, these methods are complex and molecularly ill defined. Moreover, in particular in human therapeutic applications, the presence of additional (non-human) cells during the cell culture periods is often not desired, in order to avoid contaminations with non-human cells or cellular components that might trigger e.g. immune responses in the person treated.

The present invention thus provides the advantage of providing a means to maintain hematopoietic stem and progenitor cells as a cell line that can be amplified and/or maintained for a prolonged period of time, thus providing a sufficiently high number of cells for carrying out research, such as for example research and validation studies of pharmaceutical compositions for use in the hematopoietic system or toxicity studies. Also, the hematopoietic stem and progenitor cells in accordance with the present invention may be used for cell therapy, such as in the treatment of haematological diseases, injuries or transplantations as well as for the regeneration of hematopoietic stem and/or progenitor cells after chemo- or radiotherapy applied e.g. in the context of cancer treatment. For example, the use of HSCs transplants after cancer treatments, such as irradiation or chemotherapy, has been reported to improve hematopoietic recovery (Forsberg and Smith-Berdan, 2009; Wagers, 2012).

The findings reported herein demonstrate that dermatopontin is an ECM protein that is capable of maintaining the self-renewal of highly purified HSCs ex vivo. These findings are particularly surprising, as it was previously reported that Dpt behaves differently than other ECM proteins, for example in that it reduces adhesion of whole bone marrow cells to matrix or co-cultured stroma cells in vitro (Lehrke, 2015). Furthermore, in a study related to another (unrelated) type of stem cells, i.e. mesenchymal stem cells, Dpt was found to enhance differentiation of said stem cells (Coan et al., 2014), i.e. an effect that is the opposite of the now observed maintenance of hematopoietic stem and progenitor cells when kept in culture in the presence of Dpt.

The present invention further relates to a method for maintaining hematopoietic stem and/or progenitor cells in culture, the method comprising culturing the hematopoietic stem and/or progenitor cells in the presence of dermatopontin (DPT) or a functional fragment thereof.

The definitions and preferred embodiments provided herein above with regard to the use of dermatopontin (DPT) or a functional fragment thereof for the maintenance of hematopoietic stem and/or progenitor cells in culture apply mutatis mutandis also to this method of maintaining hematopoietic stem and/or progenitor cells in culture.

In a preferred embodiment of the use or the method of the invention, DPT or a functional fragment thereof is added to the cell culture and/or DPT or a functional fragment thereof is exogenously expressed by cells present in the culture.

When DPT or a functional fragment thereof is to be added to the cell culture, the addition has to be carried out by adding DPT or a functional fragment thereof in proteinaceous form. The addition to the cell culture can be either via coating of the cell culture dishes with DPT prior to adding the hematopoietic stem and/or progenitor cells to be cultured or can be via addition of DPT to the cell culture medium. Furthermore, it is also envisaged in accordance with the present invention that the addition to the cell culture is via both (i) the coating of the cell culture dishes with DPT and (ii) the addition of DPT to the cell culture medium.

On the other hand, where DPT or a functional fragment thereof is exogenously expressed by cells present in the culture, a nucleic acid molecule encoding DPT or the functional fragment thereof and capable of expressing said protein has to be introduced into the cells.

To this end, a nucleic acid sequence encoding DPT or the functional fragment thereof can for example be incorporated into a vector, which is then introduced into the cells. Depending on the choice of vector, the nucleic acid sequence is then either stably integrated into the genome of the cells, for example via random integration or homologous recombination, or is transiently expressed from the vector, i.e. an expression vector. The nucleic acid sequence of full length human DPT is shown in SEQ ID NO:3 and the full length murine DPT is shown in SEQ ID NO:10. In addition, the nucleic acid sequence of the preferred DPT fragments discussed herein above as SEQ ID NOs:2 and 5 are represented in SEQ ID NOs: 11 and 12.

Preferably, the vector is a plasmid, cosmid, virus, bacteriophage, transposon or another vector used conventionally e.g. in genetic engineering.

The nucleic acid molecule encoding DPT or the functional fragment thereof may be inserted into several commercially available vectors. Non-limiting examples include vectors compatible with expression in mammalian cells like E-027 pCAG Kosak-Cherry (L45a) vector system, pREP (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, Okayama-Berg cDNA expression vector pcDV1 (Pharmacia), pRc/CMV, pcDNA1, pcDNA3 (Invitrogene), pSPORT1 (GIBCO BRL), pGEMHE (Promega), pLXIN, pSIR (Clontech), pIRES-EGFP (Clontech), pEAK-10 (Edge Biosystems) pTriEx-Hygro (Novagen) and pCINeo (Promega). Another vector suitable for expressing proteins in xenopus embryos, zebrafish embryos as well as a wide variety of mammalian and avian cells is the multipurpose expression vector pCS2+. For vector modification techniques, see Sambrook and Russel “Molecular Cloning, A Laboratory Manual”, Cold Spring Harbor Laboratory, N.Y. (2001).

Generally, vectors can contain one or more origins of replication (ori) and inheritance systems for cloning or expression, one or more markers for selection in the host, e.g., antibiotic resistance, and one or more expression cassettes. Suitable origins of replication include, for example, the Col E1, the SV40 viral and the M 13 origins of replication.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Such regulatory sequences are well known to those skilled in the art and include, without being limiting, regulatory sequences ensuring the initiation of transcription, internal ribosomal entry sites (IRES) (Owens, Proc. Natl. Acad. Sci. USA 98 (2001), 1471-1476) and optionally regulatory elements ensuring termination of transcription and stabilization of the transcript. Non-limiting examples for regulatory elements ensuring the initiation of transcription comprise a translation initiation codon, enhancers such as e.g. the SV40-enhancer, insulators and/or promoters, such as for example the cytomegalovirus (CMV) promoter, SV40-promoter, RSV-promoter (Rous sarcome virus), the lacZ promoter, chicken beta-actin promoter, CAG-promoter (a combination of chicken beta-actin promoter and cytomegalovirus immediate-early enhancer), the gai10 promoter, human elongation factor 1α-promoter, AOX1 promoter, GAL1 promoter CaM-kinase promoter, the lac, trp or tac promoter, the lacUV5 promoter, the autographa californica multiple nuclear polyhedrosis virus (AcMNPV) polyhedral promoter or a globin intron in mammalian and other animal cells. Non-limiting examples for regulatory elements enhancing transcriptional stability and increasing transcript levels is the woodchuck hepatitis virus post-transcriptional regulatory element (wPRE). In addition, regulatory elements ensuring transcription termination include the V40-poly-A site, the tk-poly-A site or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing, nucleotide sequences encoding secretion signals or, depending on the expression system used, signal sequences capable of directing the expressed polypeptide to a cellular compartment.

The term “expression vector”, as used herein, relates to a vector capable of directing the replication, and the expression of the nucleic acid molecule encoding DPT or the functional fragment thereof.

The co-transfection with a selectable marker such as dhfr, gpt, neomycin, hygromycin or a fluorescent protein allows the identification and isolation of the transfected cells. The transfected nucleic acid can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (GS) (Murphy et al. 1991, Biochem J. 227:277-279; Bebbington et al. 1992, Bio/Technology 10:169-175). Using these markers, the cells are grown in selective medium and the cells with the highest resistance are selected. Expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. On the other hand, expression of fluorescent proteins does not require growing cells in “selection” conditions, but isolation of transduced cells by flow cytometry.

The nucleic acid molecule encoding DPT or the functional fragment thereof may be designed for introduction into cells by e.g. chemical based methods (calcium phosphate, liposomes, DEAE-dextrane, polyethylenimine, nucleofection), non-chemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods (e.g. adenoviral, retroviral, lentiviral methods). Additionally, baculoviral systems or systems based on Vaccinia Virus or Semliki Forest Virus can also be used as vector in eukaryotic expression system for the nucleic acid molecules of the invention. Expression vectors derived from viruses such as retroviruses, vaccinia virus, adeno-associated virus, herpes viruses, or bovine papilloma virus, may be used for delivery of the nucleic acid molecules or vector into targeted cell population. Methods which are well known to those skilled in the art can be used to construct recombinant viral vectors; see, for example, the techniques described in Sambrook and Russel “Molecular Cloning, A Laboratory Manual”, Cold Spring Harbor Laboratory, N.Y. (2001) and Ausubel et al., Current Protocols in Molecular Biology, Green Publishing Associates and Wiley Interscience, N.Y. (2001). Where the nucleic acid molecules are to be introduced into the nucleus, preferred methods are e.g. microinjection or nucleofection. Methods for carrying out microinjection are well known in the art and are described for example in Nagy et al. (Nagy A, Gertsenstein M, Vintersten K, Behringer R., 2003. Manipulating the Mouse Embryo. Cold Spring Harbour, New York: Cold Spring Harbour Laboratory Press).

The nucleic acid sequence encoding DPT or the functional fragment thereof may be introduced into either the hematopoietic stem and/or progenitor cells or, where present, into feeder cells, or both. Preferably, the nucleic acid sequence encoding DPT or the functional fragment thereof is introduced into feeder cells but not into the hematopoietic stem and/or progenitor cells, in order to maintain these cells unmodified.

In a more preferred embodiment of the use or the method of the invention, the amount of DPT or of a functional fragment thereof added to the cell culture is at least 10 ng/ml. In an alternative preferred embodiment, the amount of DPT or of a functional fragment thereof added to the cell culture is at least 4.4843e-10 M.

The term “at least”, as used herein, refers to the specifically recited amount or number but also to more than the specifically recited amount or number. For example, the term “at least 10 ng/ml” encompasses also at least 20 ng/ml, at least 30 ng/ml, at least 40 ng/ml, at least 50 ng/ml, at least 60 ng/ml, at least 70 ng/ml, at least 80 ng/ml, at least 90 ng/ml, such as at least 100 ng/ml, at least 200 ng/ml, at least 500 ng/ml, at least 750 ng/ml, at least 1000 ng/ml and so on. Furthermore, this term also encompasses exactly 10 ng/ml, exactly 20 ng/ml, exactly 30 ng/ml, exactly 40 ng/ml, exactly 50 ng/ml, exactly 60 ng/ml, exactly 70 ng/ml, exactly 80 ng/ml, exactly 90 ng/ml, such as exactly 100 ng/ml, exactly 200 ng/ml, exactly 500 ng/ml, exactly 750 ng/ml, exactly 1000 ng/ml and so on.

More preferably, the amount of DPT or of a functional fragment thereof added to the cell culture is at least 1 μg/ml, more preferably at least 1.6 μg/ml, and most preferably, the amount is about 1.6n/ml.

The term “about”, as used herein, encompasses the explicitly recited amount as well as deviations therefrom of for example 15%, more preferably of 10%, and most preferably of 5%.

In another more preferred embodiment of the use or the method of the invention, the cells that exogenously express DPT or a functional fragment thereof have been modified to carry an expression construct for the expression of DPT or a functional fragment thereof.

The term “modified”, as used herein, refers to an alteration of the genetic make-up of the respective cell. In accordance with the present invention, such alterations include for example the addition of a nucleic acid sequence encoding DPT or the functional fragment thereof to the genome of the cell as well as the substitution of endogenously occurring nucleic acids within the genome of the cell by said nucleic acid sequence. In accordance with the present invention, the genetic make-up of the cell, also referred to herein as the genome of the cell relates to the entire genetic information present in the cell, including chromosomal and extra-chromosomal sequences. Accordingly, the nucleic acid sequence encoding DPT or the functional fragment thereof can be stably incorporated into a chromosome of the cell or can be present in the form of an extra-chromosomal expression vector.

The term “addition of a nucleic acid sequence” refers to the inclusion of said nucleic acid sequence into the cell's genome, without the removal of any endogenous sequences by the scientist.

A naturally occurring nucleic acid is considered to have been substituted within the genome of a cell if at least one nucleotide of the genome of the cell is replaced by the nucleic acid sequence encoding DPT or a functional fragment thereof.

Means and methods of introducing a nucleic acid sequence into a cell, and modifying the genome of said cell, have been described herein above.

In accordance with this embodiment, the cells are modified such that they carry “an expression construct for the expression of DPT or a functional fragment thereof”. Accordingly, it is required that the construct is capable of ensuring the expression of DPT or a functional fragment thereof, i.e. the nucleic acid sequence encoding the DPT or the functional fragment thereof is introduced such that it can be transcribed and translated into the corresponding (functional) DPT protein or fragment thereof. Means and methods of ensuring the expression of a target protein are well known in the art and include, without being limiting, the appropriate choice of regulatory sequences, such as e.g. translation initiation codons, enhancers, insulators, promoters, internal ribosomal entry sites (IRES) as well as regulatory elements ensuring termination of transcription and stabilization of the transcript. Such sequences and suitable vectors have been described herein above.

In another preferred embodiment of the use or the method of the invention, the DPT is from the same species as the hematopoietic stem and/or progenitor cells to be cultured.

In other words, where the hematopoietic stem and/or progenitor cells to be cultured are e.g. human cells, it is preferred that the DPT or functional fragment thereof represents the human DPT or a fragment of human DPT. Similarly, where the hematopoietic stem and/or progenitor cells to be cultured are e.g. murine cells, it is preferred that the DPT or functional fragment thereof represents the murine DPT or a fragment of human DPT.

In a further preferred embodiment of the use or the method of the invention, DPT is selected from human DPT as represented in SEQ ID NO: 1 or mouse DPT as represented in SEQ ID NO:4 or wherein the functional fragment of DPT is selected from the fragment of human DPT as represented in SEQ ID NO: 2 or the fragment of mouse DPT as represented in SEQ ID NO:5.

As is shown in the appended examples, the presence of endogenously present DPT on feeder cells is pivotal for the survival of hematopoietic stem and/or progenitor cells cultured in co-culture with said feeder cells. Moreover, as shown in example 6 below, the same beneficial effect was also found when using DPT fragments added exogenously to the cell culture medium (in the presence of 2018 stroma cells).

In accordance with the present invention, the hematopoietic stem and/or progenitor cells may further be employed in combination with additional compounds known to play a role in the maintenance of hematopoietic stem and/or progenitor cells in vitro. For example, the above discussed hematopoietic cytokines, which on their own do not provide satisfying effects, may be combined with DPT or a functional fragment thereof. Preferably, one or more cytokines selected from the group consisting of stem cell factor (SCF), thrombopoietin (TPO), angiopoietin 1 (Ang1), granulocyte colony-stimulating factor (G-CSF), Flt3-ligand (Flt3L), pleiotropin (Ptn), interleukin 3 (IL-3), interleukin 6 (IL-6), and interleukin 11 (IL-11) is employed in combination with DPT or a functional fragment thereof.

In a further preferred embodiment of the use or the method of the invention, the cell culture does not contain cells other than the hematopoietic stem and/or progenitor cells to be cultured.

In accordance with this embodiment, it is even more preferred that all cells are excluded that are not hematopoietic stem and/or progenitor cells. In other words, only the cells to be maintained in cell culture are present. Means and methods to enrich for hematopoietic stem and/or progenitor cells are well known in the art and include, without being limiting, cell sorting (flow cytometry) or magnetic-bead separation. A number of such enriched murine HSC populations have been described, such as Thy^{lo}Sca1+Lin− (Spangrude et al., 1988), cKit+Sca1+Lin− (Okada et al., 1991), side population Sca1+Lin− (Goodell et al., 1996), CD34−cKit+Sca1+Lin− (Osawa et al., 1996), CD150+CD48−cKit+Sca1+Lin− and CD150+CD48−CD41− (Kiel et al., 2005), CD45+EPCR+CD48−CD150+ (Kent et al., 2009), CD49b^{lo}RhodamineFlt3−CD34^{lo}cKit+Sca1+Lin− (Benveniste et al., 2010), etc. Lineage negative (Lin−) cells are those not expressing CD3e, CD4, CD8, CD19, B220, TER119, Gr-1, Mac-1 and where stated CD41. Lineage positive cells can be stained with the corresponding biotinylated primary antibodies and subsequently with streptavidin secondary antibodies linked with magnetic beads, therefore separated the cells from lineage negative cells (using magnets, for example BigEasy EasySep magnet, Stem Cell Technologies, Cat No 18001). Human HSCs are highly enriched in CD34+Thy1+cKit+ and lack or have a low expression of CD38, CD45RA and lineage markers (Notta et al., 2011).

In another preferred embodiment of the use or the method of the invention, the hematopoietic stem and/or progenitor cells are selected from human hematopoietic stem and/or progenitor cells obtained from bone marrow, umbilical cord blood and/or peripheral blood and/or from murine hematopoietic stem and/or progenitor cells obtained from bone marrow, yolk sac, aorta-gonad-mesonephros (AGM) region, fetal liver, spleen and/or peripheral blood.

These sources of hematopoietic stem and/or progenitor cells as well as methods of obtaining hematopoietic stem and/or progenitor cells from these sources are well known in the art. These cells may, for example, be derived from any tissue containing or expecting to contain hematopoietic stem and/or progenitor cells, such as adult bone marrow, adult spleen, mobilized peripheral blood or fetal hematopoietic sites, such as yolk sac, placenta, aorta gonad-mesonephros region, or fetal liver. In general, bone marrow cells can be obtained from crushing or flushing bones such as, but not limiting to, pelvis, ilium, femur, tibia, fibula, spine, humerus, scapula, sternum, etc., as e.g. described at the website stemcells.nih.gov/info/Regenerative_Medicine/pages/2006chapter2.aspx). Human hematopoietic stem and/or progenitor cells may be derived from similar sources, such as bone marrow, mobilized peripheral blood, cord blood. Preferably, human hematopoietic stem and/or progenitor cells are not derived from human embryos or human embryonic tissues.

In another preferred embodiment of the use or the method of the invention, the hematopoietic stem and/or progenitor cells are mammalian hematopoietic stem and/or progenitor cells.

The term “mammalian” is taxonomically well known in the art.

Preferably, the mammalian cells are derived from a mammal selected from the group consisting of e.g. human, mouse, rat, hamster, cow, cat, pig, dog, horse, rabbit or monkey. More preferably, the mammalian hematopoietic stem and/or progenitor cells are derived from human or mouse, most preferably the mammalian hematopoietic stem and/or progenitor cells are human hematopoietic stem and/or progenitor cells. As detailed herein above, the term “human hematopoietic stem and/or progenitor cells” does not encompass human embryonic stem cells.

In another preferred embodiment of the use or the method of the invention, the hematopoietic stem and/or progenitor cells have not been engineered to express (an) exogenous protein(s) other than DPT or a functional fragment thereof.

Genetical engineering of therapeutically useful cells is a common and very valuable approach in the art. Whereas it is envisaged in accordance with the present invention that the hematopoietic stem and/or progenitor cells may be modified by such genetical engineering, for example to express therapeutically relevant proteins, it is particularly preferred in accordance with this embodiment that the hematopoietic stem and/or progenitor cells have not been genetically engineered to express any exogenous protein(s), with the sole exception of DPT or a functional fragment thereof.

The term “exogenous protein” refers to a protein that is not expressed in the hematopoietic stem and/or progenitor cells that are to be cultured in the cell culture and that is experimentally introduced into said cells in order to achieve expression thereof.

To the inventor's best knowledge, DPT is not or not detectably expressed in hematopoietic stem and/or progenitor cells. In accordance with the present invention, it is thus envisaged that the hematopoietic stem and/or progenitor cells can be genetically engineered to exogenously express either DPT or a functional fragment thereof, in order to maintain the hematopoietic stem and/or progenitor cells in culture. Suitable methods for the genetic engineering of cells with a nucleic acid molecule encoding DPT or a functional fragment thereof have been discussed herein above.

In a further preferred embodiment of the use or the method of the invention, the hematopoietic stem and/or progenitor cells have not been engineered to over-express endogenously expressed proteins.

As discussed with regard to the preceding embodiment, genetical engineering of therapeutically useful cells is common in the art. In accordance with the present invention, it is generally envisaged that the hematopoietic stem and/or progenitor cells may be modified by such genetical engineering to over-express (i.e. express at higher levels) proteins that these cells already express. However, in accordance with this embodiment, it is particularly preferred that the hematopoietic stem and/or progenitor cells have not been modified to express any endogenously expressed protein(s) in a higher amount (i.e. over-express) as compared to prior to the modification. The term “endogenously expressed protein” refers to any protein that is naturally expressed in the unmodified hematopoietic stem and/or progenitor cells that are to be cultured in accordance with the present invention. As also discussed herein above, according to the present knowledge DPT is not endogenously expressed in hematopoietic stem and/or progenitor cells. However, should this knowledge turn out to be incorrect for cells obtained from certain sources, it is preferred that the hematopoietic stem and/or progenitor cells have not been engineered to over-express endogenously expressed proteins other than DPT or a functional fragment thereof.

In a further preferred embodiment of the use or the method of the invention, the hematopoietic stem and/or progenitor cells are cultured in a cell culture medium without cytokine supplementation.

The term “cytokine”, as used herein, is also well known in the art and refers to a group of cell signalling molecules including e.g. chemokines, interferons, interleukins, lymphokines and tumour necrosis factor but generally not hormones. In accordance with the present invention, the term “cytokine” also includes growth factors. Growth factors are a variety of protein molecules acting as positive regulators of cell growth and proliferation. Cytokine supplementation has been studied extensively for its effect on the maintenance of hematopoietic stem and/or progenitor cells in culture. Prominent examples include e.g. stem cell factor (SCF), thrombopoietin (TPO), angiopoietin 1 (Ang1), granulocyte colony-stimulating factor (G-CSF), Flt3-ligand (Flt3L), pleiotropin (Ptn), interleukin 3 (IL-3), interleukin 6 (IL-6), and interleukin 11 (IL-11). However, as discussed above, the results of these studies indicated that cytokine supplementation is not sufficient for a robust maintenance of hematopoietic stem and/or progenitor cells for prolonged periods.

In accordance with this preferred embodiment, the cells are cultured in a culture medium without cytokine supplementation. Preferably, the cells are cultured in a culture medium devoid of the cytokines stem cell factor (SCF), thrombopoietin (TPO), angiopoietin 1 (Ang1), granulocyte colony-stimulating factor (G-CSF), Flt3-ligand (Flt3L), pleiotropin (Ptn), interleukin 3 (IL-3), interleukin 6 (IL-6), and interleukin 11 (IL-11).

The present invention further relates to a cell culture medium for the maintenance of hematopoietic stem and/or progenitor cells, wherein the cell culture medium comprises a medium and dermatopontin (DPT) or a functional fragment thereof and further optionally comprises serum/serum replacement, (a) reducing agent(s), and/or (an) antibiotic(s).

In accordance with this embodiment, a cell culture medium is provided that is suitable for the prolonged maintenance of hematopoietic stem and/or progenitor cells in vitro or ex vivo cell culture. The essential compounds of the cell culture medium are (i) a medium and (ii) dermatopontin (DPT) or a functional fragment thereof. Suitable media as well as preferred media have been described herein above. The cell culture medium further may comprise optional compounds, such as serum/serum replacement, (a) reducing agent(s), and/or (an) antibiotic(s). Accordingly, the cell culture medium can comprise or consist of (i) a medium and (ii) dermatopontin (DPT) or a functional fragment thereof, without the presence of one or more of (iii) serum/serum replacement, (iv) (a) reducing agent(s), and/or (v) (an) antibiotic(s). The cell culture medium can also comprise or consist of:
(a): (i) a medium, (ii) dermatopontin (DPT) or a functional fragment thereof, and (iii) serum/serum replacement, wherein the cell culture medium is devoid of (iv) (a) reducing agent(s), and (v) (an) antibiotic(s);
(b): (i) a medium, (ii) dermatopontin (DPT) or a functional fragment thereof, and (iv) (a) reducing agent(s), wherein the cell culture medium is devoid of (iii) serum/serum replacement, and (v) (an) antibiotic(s);
(c): (i) a medium, (ii) dermatopontin (DPT) or a functional fragment thereof, and (v) (an) antibiotic(s), wherein the cell culture medium is devoid of (iii) serum/serum replacement and (iv) (a) reducing agent(s);
(d): (i) a medium, (ii) dermatopontin (DPT) or a functional fragment thereof, (iii) serum/serum replacement, and (iv) (a) reducing agent(s), wherein the cell culture medium is devoid of (v) (an) antibiotic(s);
(e): (i) a medium, (ii) dermatopontin (DPT) or a functional fragment thereof, (iii) serum/serum replacement, and (v) (an) antibiotic(s), wherein the cell culture medium is devoid of (iv) (a) reducing agent(s);
(f): (i) a medium, (ii) dermatopontin (DPT) or a functional fragment thereof, (iv) (a) reducing agent(s), and (v) (an) antibiotic(s), wherein the cell culture medium is devoid of (iii) serum/serum replacement;
(g): (i) a medium, (ii) dermatopontin (DPT) or a functional fragment thereof, (iii) serum/serum replacement, (iv) (a) reducing agent(s) and (v) (an) antibiotic(s).

The definitions and preferred embodiments provided herein above with regard to the use of dermatopontin (DPT) or a functional fragment thereof for the maintenance of hematopoietic stem and/or progenitor cells in culture as well as the corresponding method of the invention apply mutatis mutandis also to this cell culture medium for the maintenance of hematopoietic stem and/or progenitor cells.

The term “comprising”, as used throughout the present description, denotes that in addition to the specifically recited compound(s) or step(s), further compound(s) or step(s) may be included that have not been mentioned specifically. The term also encompasses that the composition(s), compound(s) or method(s) “consist(s) of” the specifically recited compound(s) or step(s), i.e. only the recited compound(s) or step(s) are included and no other compound(s) or step(s) are present/carried out in addition to those specifically recited herein.

The present invention further relates to a kit comprising dermatopontin (DPT) or a functional fragment thereof. Preferably, the kit comprises dermatopontin (DPT) or a functional fragment thereof, and at least one of: (a) cell culture medium; (b) one or more cytokines; (c) serum/serum replacement; (d) reducing agent(s), and/or (e) antibiotic(s). Optionally, the kit may further contain instructions for use.

The parts of the kit of the invention can be packaged individually in vials or other appropriate means depending on the respective ingredient or in combination in suitable containers or multi-container units. Manufacture of the kit preferably follows standard procedures which are known to the person skilled in the art. Whereas the term “kit” in its broadest sense does not require the presence of any other compounds, vials, containers and the like other than the recited components, the term “comprising”, in the context of the kit of the invention, denotes that further components can be present in the kit. Non-limiting examples of such further components include preservatives, buffers for storage, further cell culture supplements etc.

The kit of the invention can be used in the cell culture methods of the invention. It is particularly envisaged that the kit is a supplementation kit, i.e. that it provides the supplements required for carrying out cell cultures of hematopoietic stem and/or progenitor cells in accordance with the present invention.

All definitions and preferred embodiments provided herein above with regard to the use or the method of the invention as well as with regard to the cell culture medium of the invention, in particular preferred amounts of Dpt, preferred embodiments of the medium, cytokines, serum or serum replacement, reducing agents and/or antibiotics apply mutatis mutandis also to this embodiment.

It is of note that all the sequences accessible through the Database Accession numbers cited herein are within the scope of the present invention irrespective of whether the entry of the respective Accession No. is completely identical to the sequence displayed by the corresponding SEQ ID NO due to potential future updates in the database. Thus, this is to account for future corrections and modifications in the entries of GenBank, which might occur due to the continuing progress of science.

As regards the embodiments characterised in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims. To give a non-limiting example, the combination of claims 6 and 1 is clearly and unambiguously envisaged in view of the claim structure. The same applies for example to the combination of claims 6, 3 and 1, or the combination of claims 6, 4, 3 and 1 etc.

The figures show:

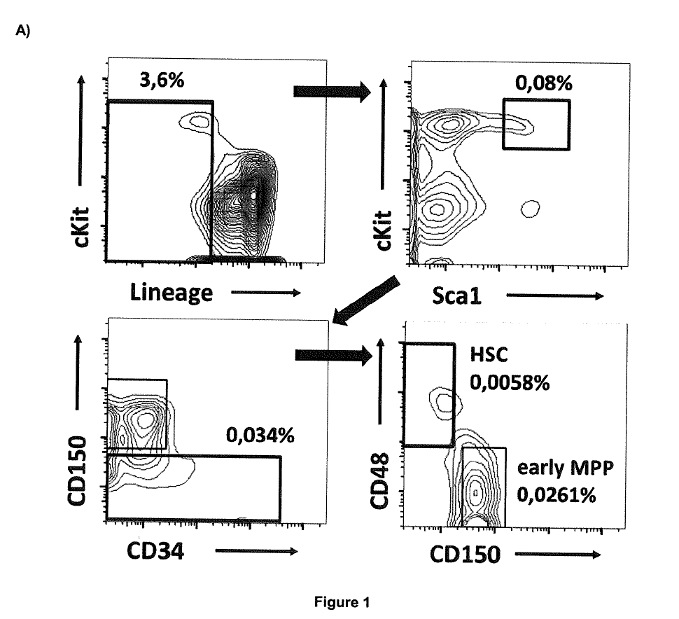

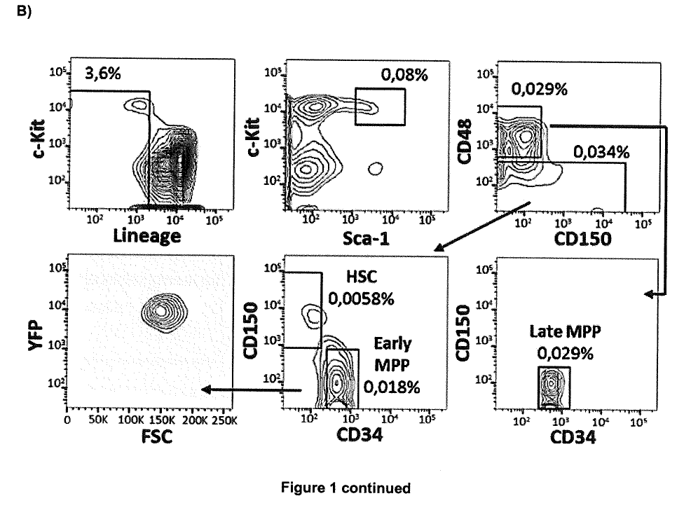

FIG. 1: Gating strategy for the isolation of HSCs and MPPs by flow cytometry. (A) Original strategy. (B) Extended strategy.

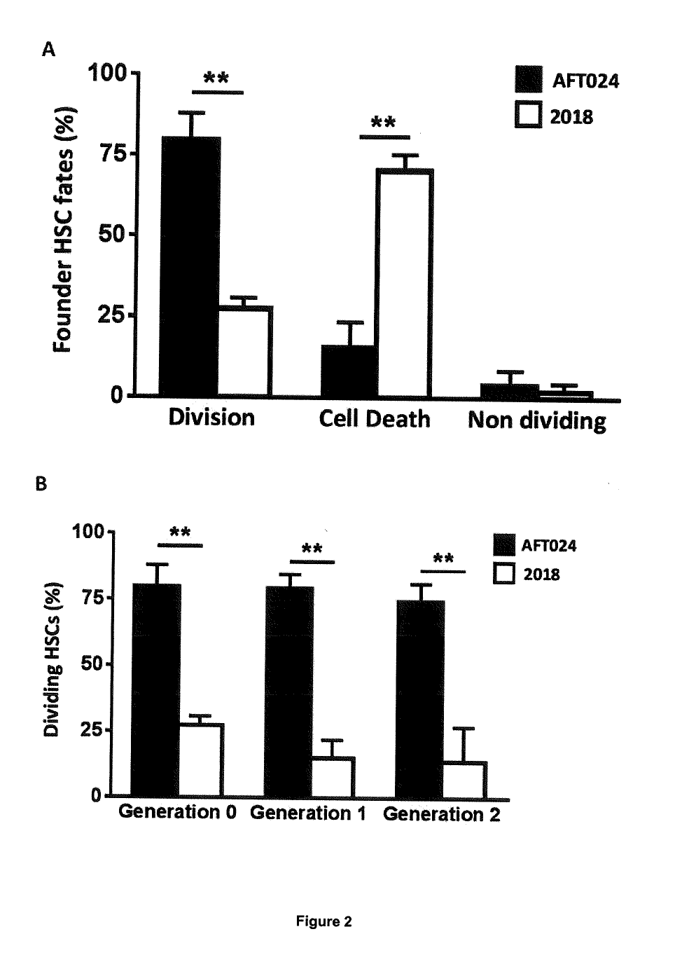

FIG. 2: Early survival/proliferation of co-cultured HSCs correlates with stroma's ability to support their ex vivo maintenance. (A) Cell-fate quantification of founder HSCs co-cultured with different stroma: supportive AFT024 (black bars, n=7 independent experiments, 290 trees) and non-supportive 2018 (white bars, n=5 independent experiments, 264 trees). (B) Quantification of dividing HSC rates on different stroma over the first three generations.

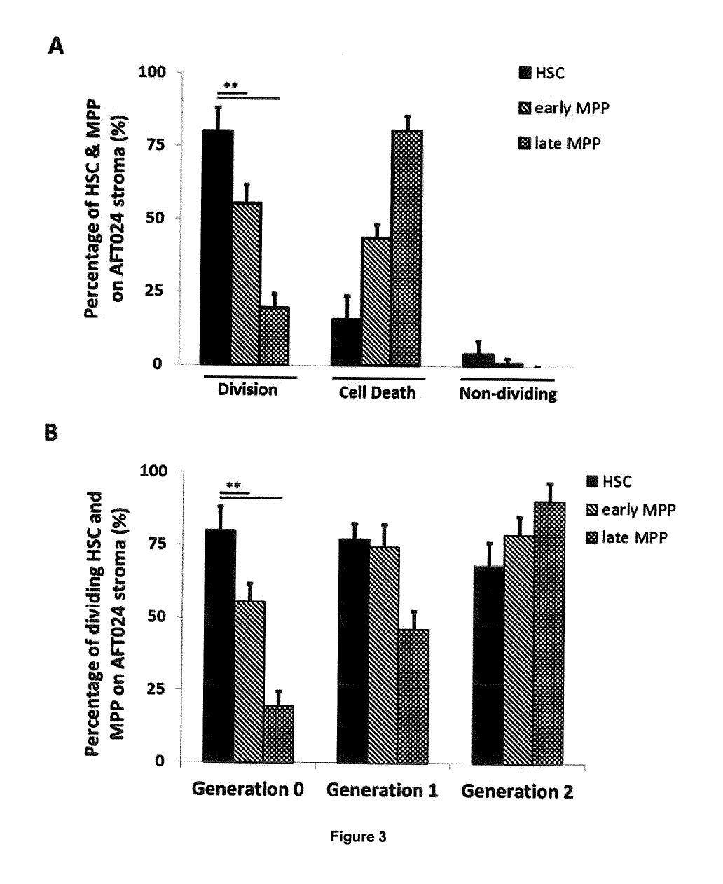

FIG. 3: AFT024 co-cultures also support survival of multipotent progenitor (MPPs) cells, after initial selection, as well as their proliferation. A) Cell-fate quantification of founder early (n=5 independent experiments, 274 trees) and late MPP (n=4 independent experiments, 211 trees) compared with HSCs cultured on supportive AFT024 stroma. (B) Quantification of dividing HSC and MPP rates on AFT024 stroma over the first three generations.

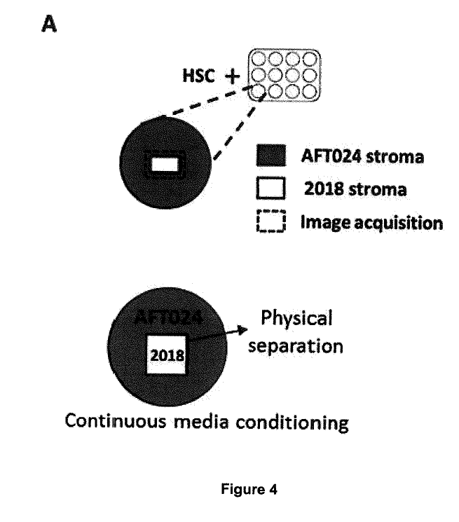

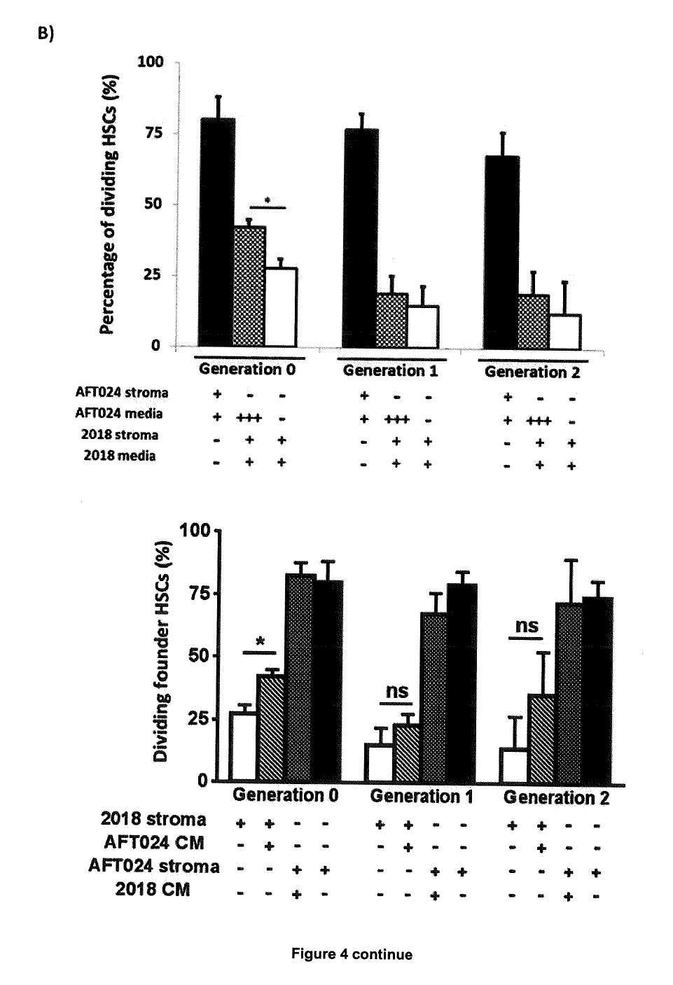

FIG. 4: Cell adhesion is the responsible mechanism for AFT024-mediated HSC maintenance ex vivo. A) Schematic representation of the experimental procedure for continuous media conditioning (upper panel): AFT024 stroma surrounding a physically separated (silicon insert) island of 2018 cells (or vice versa). Area covered by the surrounding stroma is approximately 8 times larger. HSCs were exclusively cultured in contact with the inner stroma compartment, but exposed to media mainly conditioned by the outer stroma (approximately 8× more cells). B) Generation-based analysis of dividing HSCs cultured on 2018 stroma while exposed to AFT024 conditioned media (n=3 independent experiments, 194 trees) or vice versa (n=3 independent experiments, 141 trees). White and black bars represent control conditions. Upper panel: original experiments; Lower panel: repeat experiment including additional control.

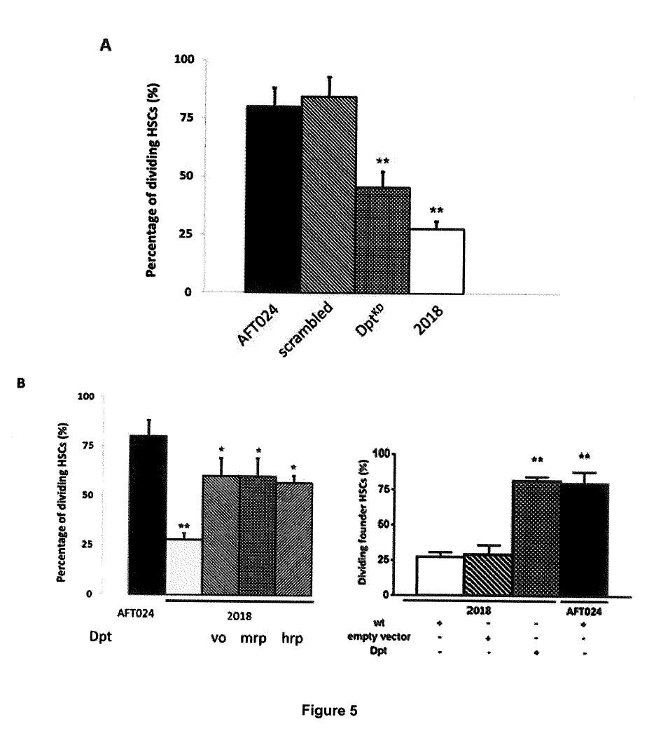

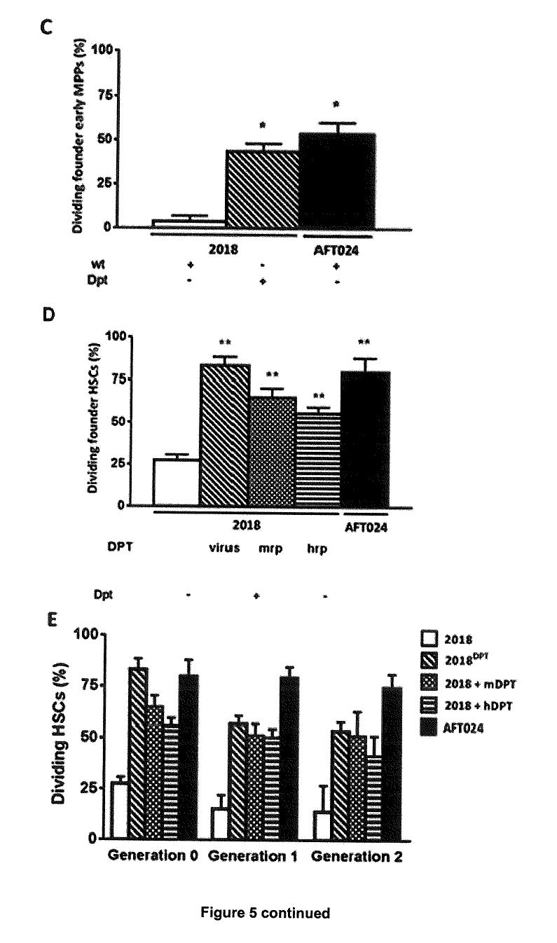

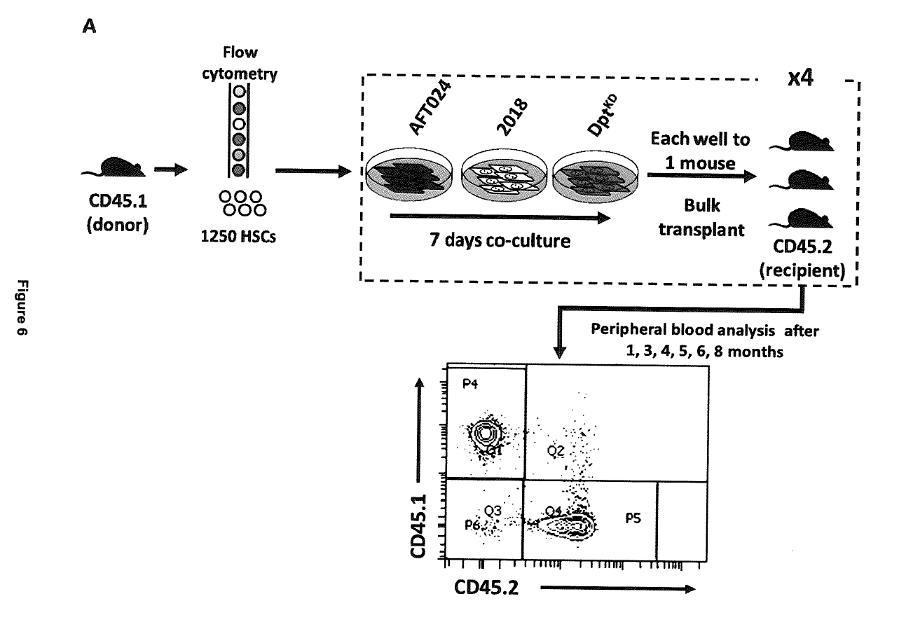

FIG. 5: Dpt is important for HSC survival and proliferation upon AFT024 co-culture, as the stroma-derived factor Dpt restores in vitro HSC/MPP behaviour under non-supportive stroma co-cultures. A) Comparison of proliferation rates of founder HSCs cultured on different stroma: wild type stroma (AFT024 black bar, 2018 white bar), AFT024 knock-down lines including scrambled shRNA control (n=3 independent experiments, 103 trees) and DPT^{KD} (99% reduced expression at RNA level, n=6 independent experiments, 211 trees). Panel (B) shows comparison of proliferation rates of founder HSCs cultured on wildtype (2018, AFT024) or virally transduced 2018 stroma overexpressing tdTOMATO−2018^{tdTOMATO} (mock, n=3 independent experiments, 120 trees) or DPT−2018^{DPT} (n=4 independent experiments, 202 trees) with initial data in the left panel, repeat experiments concerning viral ectopic expression in the right panel. (C) Similar analysis for early MPPs on wildtype (2018, AFT024) or virally transduced 2018 stroma overexpressing DPT (n=3 independent experiments, 194 trees). D) Effect of exogenous addition of 1.67 ug/ml mouse (mrp, n=4 independent experiments, 166 trees) or human recombinant DPT (hrp, n=4 independent experiments, 155 trees) on proliferation rates of founder HSCs cultured on 2018 stroma. E) Similar analysis showing the effect of exogenous DPT addition on HSC progeny over the first three cell generations.

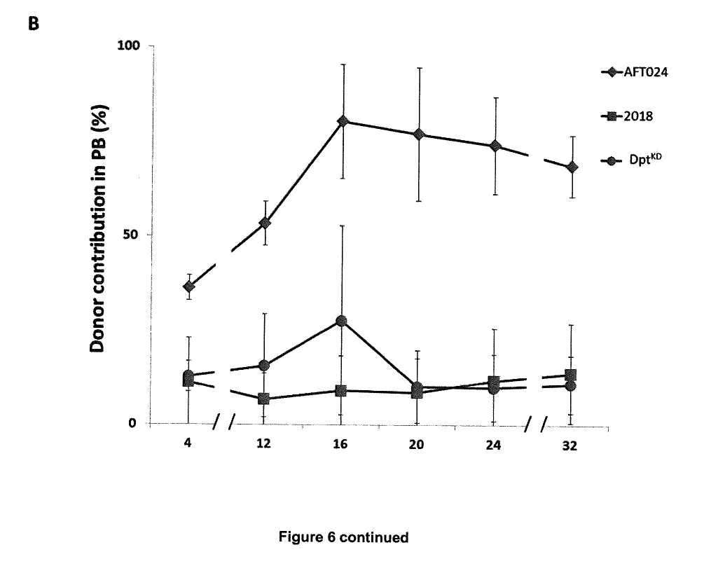

FIG. 6: Dpt is essential for ex vivo HSC maintenance. A) Experimental approach for in vivo transplantation of sorted HSCs cultured on knockdown cell lines prior to injection into sub-lethally irradiated recipients. 1250 CD45.1 HSCs were sorted and co-cultured with different stroma cell lines for seven days in vitro. Then, the content of each well was transplanted into a CD45.2 sub-lethally irradiated recipient. B) Peripheral blood (PB) contribution of donor CD45.1 cells analyzed at several time points up to 32 weeks post transplantation.

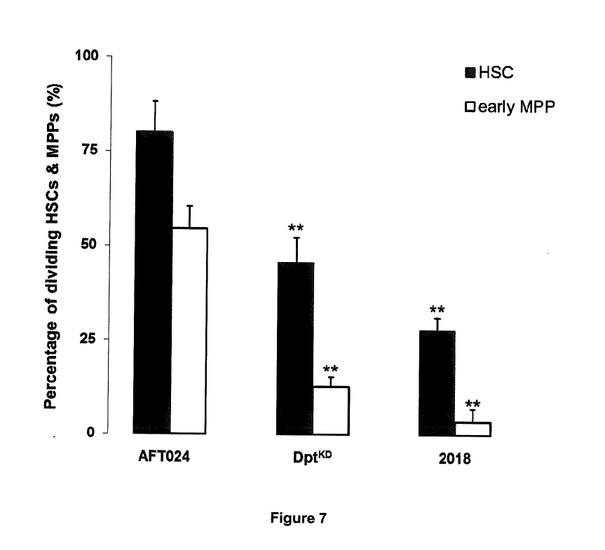

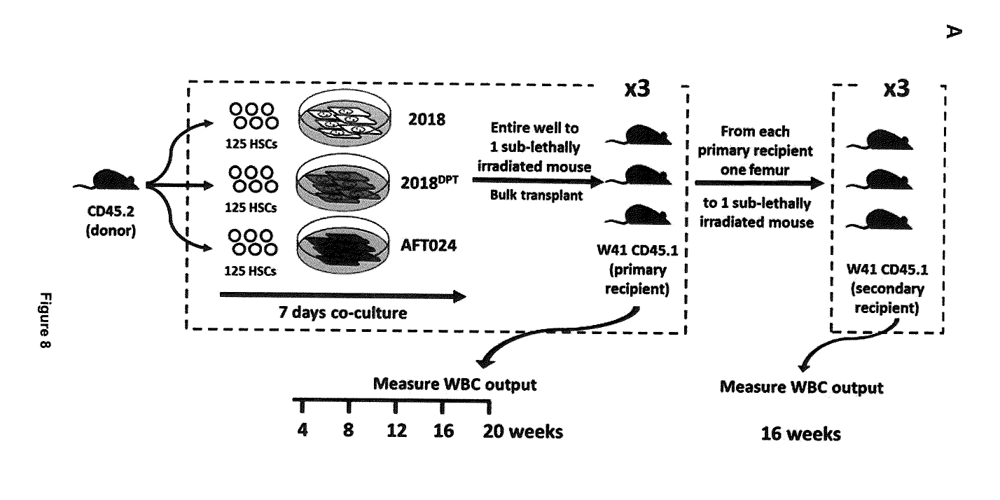

FIG. 7: Dpt is necessary for the survival and proliferation of early multipotent hematopoietic progenitors. Proliferation rates of founder HSCs or early MPPs upon co-culture with wildtype or DPT^{KD} stroma (n=3 independent experiments, 91 early MPP trees).

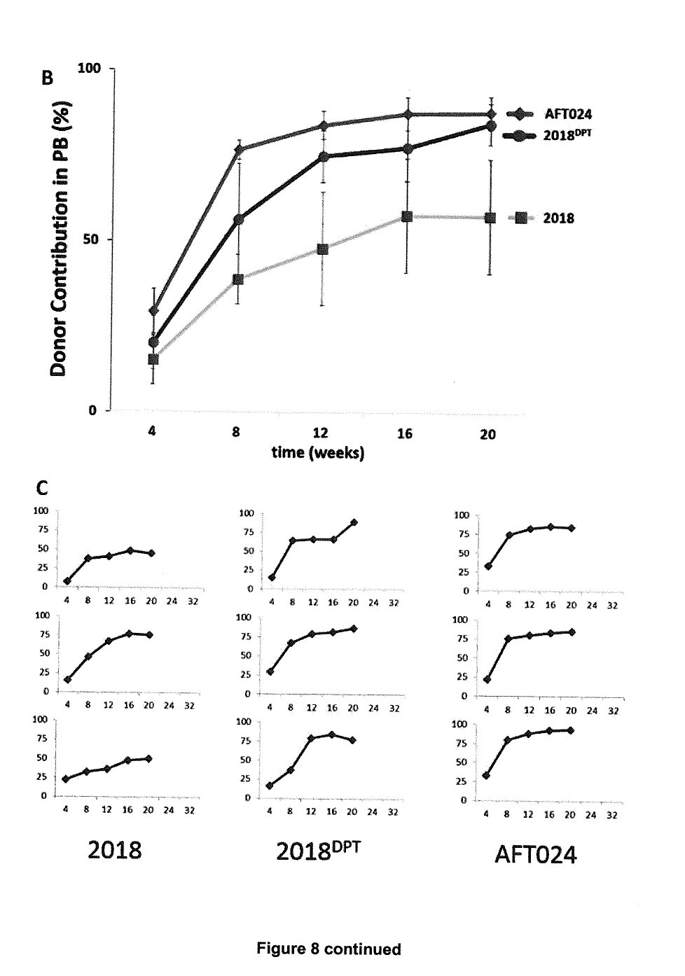

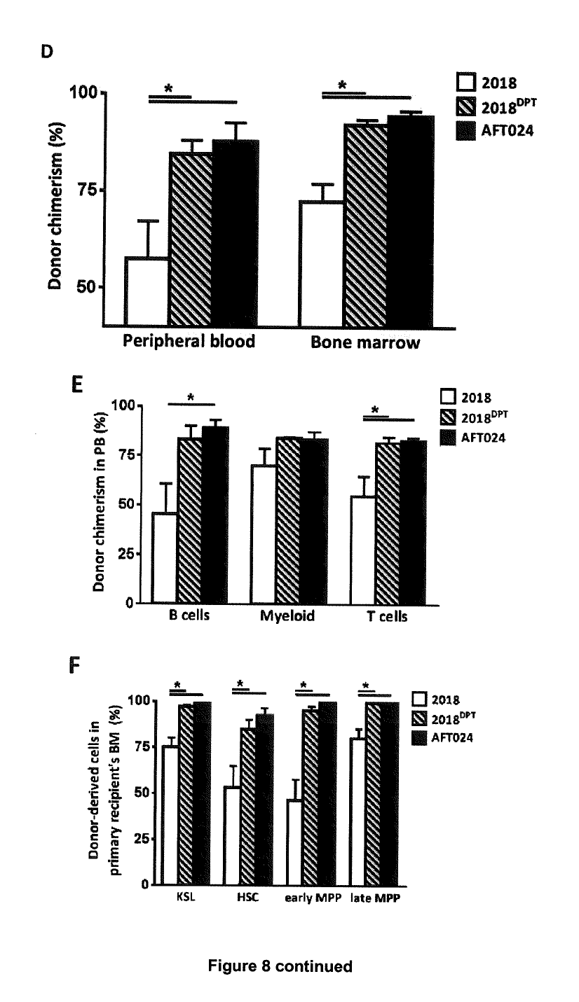

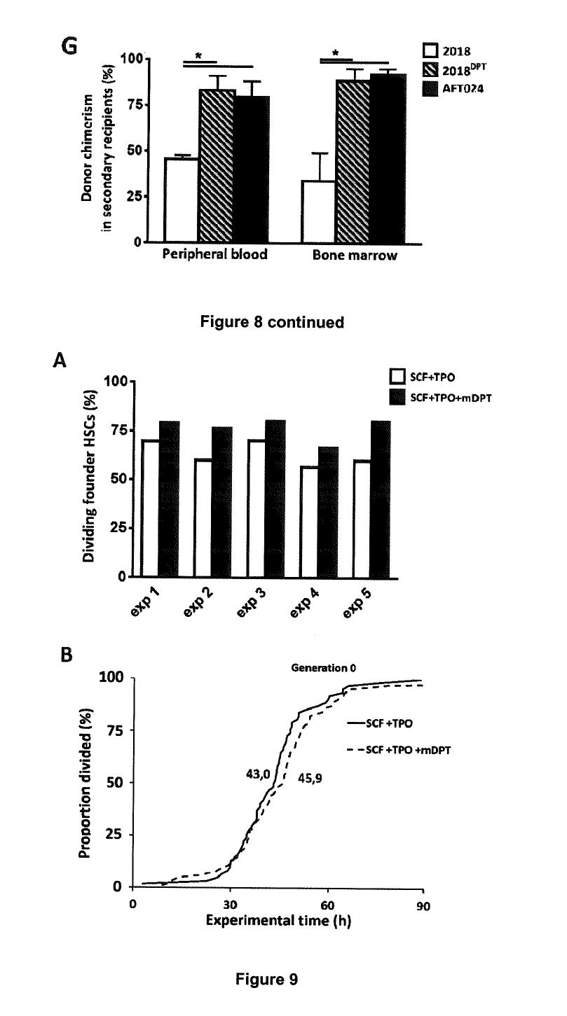

FIG. 8: Ectopic DPT expression restores long-term repopulation potential of HSCs cultured under non-supportive conditions. A) Experimental approach for in vivo transplantation of sorted HSCs cultured on genetically engineered 2018 stroma ectopically expressing DPT or wild type supportive (AFT024) and non-supportive stroma lines (2018) for seven days prior to injection into sub-lethally irradiated immunocompromised W41 recipients. Donor contribution in peripheral blood was analyzed at several time points up to 20 weeks post-transplantation and plotted as the average of all recipients per condition (B) are for each individual recipient separately (C). D) Donor contribution was calculated in the peripheral blood (PB) and bone marrow (BM) 20 weeks post transplantation. E) Lineage-specific donor contribution in recipients' peripheral blood 20 weeks post-transplant. (F) Cell type-specific contribution of donor cells in recipient's bone marrow. (G) For secondary transplantations, total bone marrow cells of one complete femur from each primary recipient were injected into sub-lethally irradiated W41 mice used as secondary recipients. Donor contribution was calculated in the peripheral blood (PB) and bone marrow (BM) 16 weeks after serial transplantation.

FIG. 9: Exogenous addition of recombinant DPT improves HSC clonogenicity under stroma/serum-free conditions. A) Founder HSC proliferation rates in stroma/serum-free cultures supplemented with 100 ng/ml SCF, 100 ng/ml TPO without (n=5 independent experiments, at least 30 trees per experiment, 153 trees total) or with 1.67 μg/ml mouse DPT (n=5 independent experiments, at least 30 trees per experiment, 190 trees total). B) HSC proliferation kinetics in stroma/serum-free conditions in the presence of 100 ng/ml SCF, 100 ng/ml TPO and 1.67 μg/ml mDPT. Values indicate the time at which 50% of the cells have divided. Dividing cells from three independent experiments were pooled.

The examples illustrate the invention:

### EXAMPLE 1: METHODS AND MATERIALS

### Mouse Strain

Transgenic mice (B6J; 129-Tg(CAG-EYFP)7AC5Nagy/J) expressing the yellow fluorescent protein (YFP) under the control of the chicken beta actin promoter coupled with the cytomegalovirus (CMV) immediate early enhancer (Hadjantonakis et al., 2002) were backcrossed with C57Bl/6J mice for at least 10 generations and were used in the present study. Wild type C57Bl/6J-Ly5.2, C57Bl/6J-Ly5. I or immunocompromised C57BL6J-Gpi1a Ptprca KitW-41JJ mice were used in transplantation experiments.

### Hematopoietic Stem Cell (HSC) Isolation & Mouse Preparation

Murine femur, tibia and pelvis were isolated and washed in Dulbecco's phosphate buffer saline DPBS (Gibco, Cat No 14190-169). Residual tissues and muscles were removed from isolated bones which were then crushed. Cell suspensions were filtered through a 100 μm cell strainer (Schubert and Weiss, Cat No FALC352360) and then centrifuged at 1000 rpm for 5 minutes at 4° C. The supernatant was decanted and the cell pellet was re-suspended in (2.5 ml per mouse) FACS buffer (1 mM EDTA, 5% FCS in PBS). Biotinylated lineage antibodies, such as CD3e (eBioscience, clone 145-2C11, Cat No 13-0031-85), B220 (eBioscience, clone RA3-6B2, Cat No 13-0452-86), CD19 (eBioscinese, clone eBio1D3, Cat No 13-0193-85), CD41 (eBioscience, clone eBioMWRag30, Cat No 13-0411-85), Terr119 (eBioscience, clone TER-119, Cat No 13-5291-85), Gr1 (eBioscience, clone RB6-8C5, Cat No 13-5931-85) and Mac1 (eBioscience, clone M1/70, Cat No 13-0112-85) were incubated at a dilution of 1:100 for 20 minutes on ice. The cell solution was centrifuged again and re-suspended as mentioned above. Streptavidin-labelled magnetic beads (Roth, Cat No HP571) were incubated for 20 minutes on ice. Cells were then transferred to a polypropylene round bottom (PP) tube (BD Falcon, product no 352063) and placed in a magnet (BigEasy EasySep magnet, Stem Cell Technologies, Cat No 18001) for 7 minutes, to separate lineage positive (Lin^{pos}) from low/negative (Lin^{neg}) cells. Cells were centrifuged and re-suspended in FACS buffer. Finally, the antibodies for the HSC staining were added (CD150, CD48, cKit, Scab, CD34 and Streptavidin directly conjugated antibodies) based on the cell number (0.2 μl Ab per 10⁶ cells, except for CD34 for which 25 μl were added per mouse, but not more than 50 μl in total) and incubated for 30-45 minutes on ice. The cell solution was washed, centrifuged and re-suspended in FACS buffer (≈250 μl per mouse) and filtered before flow cytometry analysis and cell sorting. Lin^{pos} cells were used for single-stained/compensation.

### Flow Cytometry

The flow cytometry analysis was conducted on a FACS Aria I and later in a FACS Aria III machine (Beckton & Dickinson, Cat No 648282) equipped with a 405 nm violet laser, a 488 nm blue laser, 561 nm yellow/green laser and a 633 nm red laser. Bone marrow cells were sorted using the 70 mm nozzle, whereas stroma cells were sorted with the 100 mm nozzle. For multicolour, flow-cytometric analysis and sorting, single-stained samples were used to calculate the bleed-through of each fluorochrome to all other channels. HSCs and hematopoietic multipotent progenitor (MPP) populations were isolated based on the scheme shown in FIG. 1.

### Stroma Cell Culture & Preparation of Co-Culture Monolayers

Stroma cell lines AFT024, 2012 and 2018 isolated from the fetal liver of E14.5 mice (Wineman et al., 1996) were used. Cells were cultured in high glucose DMEM (Gibco, Cat No 11960-085) supplemented with 10% FCS 14 (PAA, Cat No A15-101, Lot No. A10108-2429), 0.1 mM non-essential amino acids 100× (Gibco, Cat No 11140-035), 1 mM sodium pyruvate (Sigma, Cat No S8636), 2 mM L-glutamine (Gibco, Cat No 25030-024), 5×10⁻⁵M β-Mercaptoethanol (Sigma, Cat No M3142-25ML) and penicillin/streptomycin (Gibco, Cat No 15140-122) at 33° C. with 100% humidity and 5% CO₂. Stroma was trypsinised by adding 0.05% trypsin-EDTA (Gibco, Cat No 25300-096) for 2-3 minutes.

Before the co-culture of freshly purified HSCs with stroma cells, the stroma cells were plated in monolayers and irradiated with 20 Gy using a Co source (Gammacell II, Model GC 220 Type B, Cat No CDN-U13). Before the co-culture, the media was exchanged with “modified Dexter media” (high glucose DMEM (Gibco, Cat No 11960-085), 10% ml FCS 14 (PAA, Cat No A15-101, Lot No. A10108-2429), 10% Horse serum (Gibco, Cat No 16050-122, Lot No 460470), 5×10⁻⁵M β-mercaptoethanol, 10⁻⁶M Hydrocortisone (Stem Cell Technologies, Cat No 07904) and penicillin/streptomycin (Gibco, Cat No 15140-122).

### Time-Lapse Imaging

Time-lapse imaging experiments were conducted using the Zeiss Axiovert 200M or AxioObserver.Z1 microscopes equipped with motorised stages for multi-positional acquisition. Endogenous YFP signal was detected using the Zeiss Filter 46HE filter (Zeiss, Excitation BP500/25 DMR 25, Beam Splitter FT 515HE, Emission 535/30 DMR 25, Cat No 489046-9901-000). Phase-contrast or bright field pictures were acquired every 6 to 12 minutes and fluorescent pictures every 15 minutes using a 5×Plan NeoFluar (numerical aperture 0.3), and recorded by an AxioCamHRm camera (at 1388×1040 or 692×520 pixel resolution) using the Zeiss AxioVision 4.8 software or later. Mercury lamps (HXP or HBO, both Osram) or light-emitting diode based systems (LEDs, Lumencore, Laser 2000, Cat No 1303749) were used for fluorescent illumination.

Dermatopontin shRNA, cDNA and Protein

Dpt-specific shRNA was taken from the RNAi consortium shRNA library database (see the world wide web at broadinstitute.org/rnai/trc/lib) and has the hairpin sequence: 5′-CCGGGCGAGGAGCAACAACCACTTTCTCGAGAAAGTGGTTGTTGCTCCTCGCTTTTTG-3′ (SEQ ID NO:7).

Mouse Dpt cDNA sequence was isolated from AFT024 after RNA extraction and PCR using a forward PCR primer having the sequence 5′-CACGGATCCGCCACCATGGACCTCACTCTTCTGTGGGTTC TTCTGCCACTGG-3′ (SEQ ID NO:8) and a reverse PCR primer with the sequence: 5′-CACGGATC CCTAAACGTTTTCGAATTCGCAGTCG-3′ (SEQ ID NO:9).

Recombinant mouse DPT (SEQ ID NO:5) was obtained from R&D Systems (Catalog Number: 5749-DP-050) and represents a DPT fragment consisting of amino acids 19 to 201 of mouse DPT represented by UniProt accession number Q9QZZ6 (last modified Jul. 9, 2014).

Recombinant human DPT (SEQ ID NO:2) was obtained from R&D systems (Catalog Number: 4629-DP-050) and represents a DPT fragment consisting of amino acids 19 to 201 of human DPT represented by UniProt accession number Q07507 (last modified Sep. 3, 2014).

### Single-Cell Tracking & Statistical Analysis

Single cell tracking and reconstruction of cells' genealogy into trees was performed manually using the software TTT (Eilken et al., 2009; Rieger et al., 2009). Results were analyzed using the non-parametric Mann-Whitney test (one-tailed) for data not following the Gaussian distribution, unless otherwise mentioned. Error bars are standard deviation (SD).

### EXAMPLE 2: AFT024-BASED CONDITIONS PROMOTE SURVIVAL OF CO-CULTURED HSCS

The fetal-liver derived stroma cell line AFT024 is capable of maintaining HSC numbers in long-term co-cultures, whereas the 2018 line (derived from the same experiment) failed (Moore et al., 1997). These findings suggest that HSCs co-cultured with the different stroma cells follow distinct cell fates. However, so far, studies analysing the composition of these cultures failed to identify the principle underlying this environment-specific HSC behaviour.

Continuous time-lapse imaging revealed that the majority of HSCs (founder HSCs) co-cultured with AFT024 divided (80.0%±8.0%), whereas 15.8%±7.8% died directly after isolation (generation 0). On the contrary, in conditions not supporting HSC maintenance (2018 stroma), only a small proportion of HSCs divided (26.2%±4.0), whereas the majority died (71.6%±5.8%), as shown in FIG. 2A. In both cases, around 5% of single HSCs survived without division until the end of the time-lapse movie. (FIG. 2B).

### EXAMPLE 3: EARLY SURVIVAL CORRELATES WITH THE PRIMITIVENESS OF THE CO-CULTURED HEMATOPOIETIC POPULATION

To assess the effect of AFT024 co-culture on the less primitive hematopoietic multipotent progenitors (MPPs), two different populations were isolated and co-cultured with the fetal-liver stroma. Early MPPs were identified as CD150⁺CD48⁻CD34⁺ KSL and late MPPs as CD150⁻CD48⁺CD34⁺ KSL. Co-culture with AFT024 stroma resulted in 54.5%±6.0% of early MPPs dividing and 45.1%±5.2% dying (FIG. 3A). In contrast, only 18.6%±16.8% of the more differentiated MPPs (late MPPs) divided on AFT024, with the remaining 81.5%±16.8% dying. The majority of early and late MPPs died at the initial generation after isolation, when co-cultured with the 2018 stroma (data not shown).

Analysis of later generations showed elevated levels of division (76.6%±7.0% of generation 1 and 80.9%±8.1% of generation 2) for the early MPPs compared to generation 0 (54.5%±6.0%). The progeny of late MPPs also showed higher divisional rates (in terms of proliferation), with 73.6% (±30.7%) and 88.2% (±11.5%) of cells dividing in generation 1 and 2 respectively (FIG. 3B). These data suggest that an early selection mechanism based on the primitiveness of the co-cultured hematopoietic cell populations exists, whereas the progeny of surviving cells is then supported by AFT024 co-cultures. Interestingly, late MPPs require two generations to reach the survival/proliferation level of HSC and early MPP progeny.

### EXAMPLE 4: CELL ADHESION IS THE PREDOMINANT MECHANISM FOR AFT024-MEDIATED HSC MAINTENANCE

To investigate a potential effect from AFT024 secreted factors, HSCs were cultured on 2018 stroma, while being exposed to media stably conditioned by AFT024 cells. The two different stroma lines were physically separated by a silicon insert (FIG. 4A). It is important to note that the inner surface of the silicon insert is 0.42 cm², while the culture area of the entire well (of a 12-well plate) is around 8 times bigger (3.5 cm²).

HSCs (founder HSCs) cultured in contact with 2018 stroma while being exposed to AFT024-conditioned media showed a 1.5-fold increase in survival/proliferation (42.1%±2.7% versus 27.9%±3.2% in the original control experiment and 42.1%±2.7% versus 27.5%±3.3% in the repeat control experiment; FIG. 4B) and reduced levels of cell death (data not shown). However, further analysis of later generations showed no change in the levels of division compared to the control, suggesting that factor(s) secreted by AFT024 have only a transient positive effect upon HSC proliferation and that cell adhesion is the predominant mechanism responsible for HSC maintenance. Stroma-free cultures led to death of HSCs (data not shown), further supporting the importance of adhesion molecules.

### EXAMPLE 5: CONFIRMATION OF GENE DIFFERENTIAL EXPRESSION BY QUANTITATIVE RT-PCR

The lists of genes preferentially or exclusively expressed on the AFT024 stroma were identified using subtractive libraries (Hackney et al., 2002) and micro-array analysis (Charbord and Moore, 2005). In general, high-throughput approaches lack sensitivity of complementary methods, such as the quantitative real-time PCR (qRT-PCR). For this reason, confirmation of the differential expression of genes matching the time-lapse imaging observations (transmembrane, cell surface or extracellular matrix molecules promoting survival/proliferation or block apoptosis/differentiation) was necessary, prior to the molecular manipulation of the AFT024 supportive stroma.

Intron-separated (if applicable) gene-specific PCR primers were designed for each gene of interest (170 genes checked in total). For this analysis, non-irradiated AFT024 stroma cells were compared with 2018, mainly for the expression of membrane-bound, extra-cellular matrix (ECM) and cell adhesion molecules. qRT-PCR experiments confirmed 33 out of the 170 tested genes (approximately 20%) published to be preferentially or exclusively expressed by the AFT024 stroma. Irradiated stroma cells were also analyzed to better mimic the co-culture conditions.

### EXAMPLE 6: DERMATOPONTIN (DPT) IS ESSENTIAL FOR THE SURVIVAL/PROLIFERATION OF HSCS CULTURED UNDER AFT024 CONDITIONS, AS THE STROMA-DERIVED FACTOR DPT RESTORES IN VITRO HSC/MPP BEHAVIOUR UNDER NON-SUPPORTIVE STROMA CO-CULTURES

To assess the effect of differentially expressed genes, Dpt-specific shRNA was generated and introduced into AFT024 stroma cells through viral vectors (lentiviruses). Dpt knock-down AFT024 stroma (Dpt^{KD}) showed reduced potential to support HSC survival in vitro. In detail, 45.6% of HSCs co-cultured with Dpt^{KD} divided compared to 80.3% in the wild type AFT024 showing a 1.8 fold reduction (FIG. 5A). Notably, AFT024 stroma transduced with scrambled shRNA had no influence on the survival/proliferation of co-cultured HSCs.

Conversely, initial experiments revealed that Dpt viral over-expression (the DPT overexpression vector is shown in SEQ ID NO:6) on 2018 stroma resulted in a 2-fold increase of HSC survival of generation 0 cells (60.2%±8.9% versus 27.9%±3.2% on wild type 2018) (FIG. 5B, left panel). Repeated experiments (FIG. 5B, right panel) confirmed that ectopic DPT expression (the DPT expression viral vector is shown in SEQ ID NO:6) on 2018 stroma (2018^{DPT}) restored founder HSCs' proliferation to AFT024 levels (81.5%±2.9%, FIG. 5B). Similar effects were observed on the proliferation levels of freshly isolated early MPPs (43.6%±4.7%, FIG. 5C).

Exogenous addition of recombinant mouse DPT (5 μg) in HSC co-cultures with 2018 showed a similar potential in rescuing HSC survival/proliferation as viral over-expression. Recombinant DPT was exogenously added to non-supportive co-cultures with 2018 stroma, resulting in a 2.4-fold increase of HSC survival/proliferation (FIG. 5D). Interestingly, human recombinant DPT was equally capable of rescuing survival/proliferation of murine HSCs (2-fold increase). Cell-fate analysis of HSC progeny revealed higher levels of cell divisions in all non-supportive conditions in the presence of DPT (viral expression or exogenous addition of recombinant proteins, FIG. 5E).

### EXAMPLE 7: DPT IS NECESSARY FOR HSC SURVIVAL AND MAINTENANCE

To confirm the role of DPT in the ex vivo maintenance of HSCs, in vivo transplantation experiments were performed. Freshly purified HSCs from CD45.1 mice were co-cultured with wild-type or Dpt^{KD} stroma cells for 7 days. Then, their progeny was transplanted into sub-lethally irradiated CD45.2 recipient mice (FIG. 6A).

The analysis of peripheral blood from the CD45.2 recipients resulted in a clear engraftment difference from cells cultured on AFT024 or 2018. HSCs and their progeny that were cultured on AFT024 stroma initially resulted in a 36% donor contribution, whereas cells cultured on 2018 or Dpt^{KD} stroma in 11.3% and 12.9% respectively (4 weeks). At 16 weeks, the contribution from AFT024-cultured cells increased to 80.5%, while 2018-cultured cells remained at 9.1% and Dpt^{KD}-cultured cells transiently increased to 27.7%. Later time points linked to engraftment by long-term HSCs showed that AFT024-cultured cells had an average contribution of 73.4%, with those cultured on 2018 contributing approximately 10%. Interestingly, cells cultured on Dpt^{KD} stroma had similarly low engraftment as those cultured on 2018 (average of 11%, FIG. 6B) showing a 7-fold reduced HSC potential. These data confirm that DPT is essential for the ex vivo maintenance of HSCs under AFT024-based co-cultures. Overall, these data show that Dpt is a novel molecule that can be used to maintain HSC ex vivo.

### EXAMPLE 8: DPT IS NECESSARY FOR THE SURVIVAL AND PROLIFERATION OF EARLY MULTIPOTENT HEMATOPOIETIC PROGENITORS

To further investigate the specificity of the negative effect of the knocked-down AFT024 stroma upon the survival/proliferation of co-cultured HSCs, we tested the effect of manipulated stroma upon early MPPs. As shown in FIG. 3A, AFT024 stroma also supports survival/proliferation of co-cultured MPPs, since 54.5% (±6.0%) of the cells divided compared to 80.0% (±8.0%) of HSCs, in generation 0 (founder MPPs).

Comparison of the survival/divisional rates between HSCs and early MPPs co-cultured with the DPT^{KD} stroma (FIG. 7) showed a 1.75-fold decrease for HSCs (from 80.0%±8.0% on AFT024 to 45.6%±6.5% on knocked-down AFT024) and a 4.25-fold decrease for early MPPs (from 54.5%±6.0% to 12.8%±2.6% on knocked-down AFT024). These data illustrate that DPT is important for the survival of both HSCs and early MPPs.

### EXAMPLE 9: ECTOPIC DPT EXPRESSION RESTORES THE EFFECTS OF NON-SUPPORTIVE STROMA ON LONG-TERM REPOPULATING CELLS

In order to investigate the effect of ectopic DPT expression on repopulating cells in vivo, HSCs were co-cultured with manipulated 2018^{DPT} or wildtype (AFT024, 2018) stroma for seven days before being transplanted to sub-lethally irradiated immunocompromised W41 recipients (FIG. 8A). Equally high chimerism (percentage of donor-derived cells) was achieved in all primary and secondary recipients transplanted with AFT024 or manipulated 2018^{DPT} co-cultured cells, compared to the significantly lower contribution of 2018 co-cultured cells over time (FIG. 8B-C, 8G). Five months post transplantation (a time point commonly linked with contribution from long-term HSCs), the vast majority of cells in the peripheral blood of recipients transplanted with AFT024 or 2018^{DPT} co-cultured cells were donor-derived (84.5% and 87.7% respectively) compared to almost half (57.7%) from 2018 co-cultured cells. Similar differences were also observed in the bone marrow compartment of primary recipients at the same time point (FIG. 8D). Serial transplantation experiments revealed that donor contribution in the peripheral blood and bone marrow of secondary recipients was also significantly higher in the AFT024 and 2018^{DPT} conditions compared to 2018 (16 weeks timepoint, FIG. 8G). Analysis of different blood lineages revealed marked reduction in B- and T-cell contribution from 2018 co-cultured HSCs (FIG. 8E), as well as reduced number of immunophenotypic HSC and MPPs in bone marrow (FIG. 8F).

### EXAMPLE 10: EXOGENOUS ADDITION OF RECOMBINANT DPT IMPROVES HSC CLONOGENICITY UNDER DEFINED STROMA/SERUM-FREE CULTURE CONDITIONS

Culturing HSCs under stroma- and serum-free conditions leads to quick loss of their self-renewal due to extensive death and/or differentiation. Addition of stem cell factor (100 ng/ml SCF) and thrombopoietin (100 ng/ml TPO) has been reported to support short-term self-renewal (Ema et al., 2000). We therefore sought to investigate the effect of supplementing those defined conditions with 1.67 μg/ml recombinant murine DPT. To this end, plates were coated with 0.1% gelatin for more than one hour at 37° C. The factors SCF, TPO and DPT were added to serum-free medium (StemSpan SFEM) and the thus supplemented medium was added to the coated wells of the cell culture plates. Subsequently, HSCs were added to the wells of the cell culture plates.

In all experiments, the number of proliferating founder HSCs was increased in the presence of recombinant DPT by 10-20% (FIG. 9A) without affecting cell-cycle progression (FIG. 9B). These results illustrate that DPT also enhances HSC clonogenicity in the absence of serum and stroma, and therefore, could supplement standard cytokine cocktails used for short-term HSC culture.

### REFERENCES

Aggarwal, R., Lu, J., Pompili, V. J., and Das, H. (2012). Hematopoietic stem cells: transcriptional regulation, ex vivo expansion and clinical application. Curr. Mol. Med. 12, 34-49.
Antonchuk, J., Sauvageau, G., and Humphries, R. K. (2002). HOXB4-induced expansion of adult hematopoietic stem cells ex vivo. Cell 109, 39-45.
Baum, C. (2004). Chance or necessity? Insertional Mutagenesis in Gene Therapy and Its Consequences. Mol. Ther. 9, 5-13.
Benveniste, P., Frelin, C., Janmohamed, S., Barbara, M., Herrington, R., Hyam, D., and Iscove, N. N. (2010). Intermediate-term hematopoietic stem cells with extended but time-limited reconstitution potential. Cell Stem Cell 6, 48-58.
Bodine, D., Karlsson, S., and Nienhuis, A. (1989). Combination of interleukins 3 and 6 preserves stem cell function in culture and enhances retrovirus-mediated gene transfer into hematopoietic stem cells. Proc. Natl Acad. Sci. USA 86, 8897-8901.
Bodine, D. M., Orlic, D., Birkett, N.C., Seidel, N. E., and Zsebo, K. M. (1992). Stem cell factor increases colony-forming unit-spleen number in vitro in synergy with interleukin-6, and in vivo in Sl/Sld mice as a single factor. Blood 79, 913-919.
Challen, G. a., Boles, N., Lin, K. K., and Goodell, M. a. (2009). Mouse Hematopoietic Stem Cell Identification And Analysis. Cytom. A 75, 14-24.
Charbord, P., and Moore, K. (2005). Gene Expression in Stem Cell-Supporting Stromal Cell Lines. Ann N Y Acad Sci. 1044, 159-167.
Chen, X., Dusevich, V., Feng, J., Manolagas, S., and Jilka, R. (2007). Chen, X.-D., Dusevich, V., Feng, J. Q., Manolagas, S. C. & Jilka, R. L. Extracellular matrix made by bone marrow cells facilitates expansion of marrow-derived mesenchymal progenitor cells and prevents their differentiation into osteoblasts. J. Bone Miner. Res. 22, 1943-1956.
Coan, H. B., Lively, M. O., and Van Dyke, M. E. (2014). Dermatopontin in the extracellular matrix enhances osteogenic differentiation of adipose-derived mesenchymal stem cells. Musculoskelet. Biol. 1.
Eilken, H. M., Nishikawa, S.-I., and Schroeder, T. (2009). Continuous single-cell imaging of blood generation from haemogenic endothelium. Nature 457, 896-900.
Ema, H., Takano, H., Sudo, K., and Nakauchi, H. (2000). In vitro self-renewal division of hematopoietic stem cells. J Exp Med. 192, 1281-1288.
Feng, Q., Chai, C., Jiang, X., Leong, K., and Mao, H. (2006). Expansion of engrafting human hematopoietic stem/progenitor cells in three-dimensional scaffolds with surface-immobilized fibronectin. J Biomed Mater Res A. 78, 781-791.
Forbes, E. G., Cronshaw, D., MacBeath, J. R., and Hulmes, D. J. (1994). Tyrosine-rich acidic matrix protein (TRAMP) is a tyrosine-sulphated and widely distributed protein of the extracellular matrix. FEBS Lett. 351, 433-436.
Forsberg, E. C. and Smith-Berdan, S. (2009). Parsing the niche code: the molecular mechanisms governing hematopoietic stem cell adhesion and differentiation. Haematologica. 94(11):1477-81.
Goodell, B. M. A., Brose, K., Paradis, G., Conner, A. S., and Mulligan, R. C. (1996). Isolation and Functional Properties of Murine Hematopoietic Stem Cells that are Replicating In Vivo. J. Exp. Med 183, 1797-1806.
Hackney, J., Charbord, P., Brunk, B. P., Stoeckert, C. J., Lemischka, I. R., and Moore, K. a (2002). A molecular profile of a hematopoietic stem cell niche. Proc Natl Acad Sci USA. 99, 13061-13066.
Hadjantonakis, A.-K., Macmaster, S., and Nagy, A. (2002). Embryonic stem cells and mice expressing different GFP variants for multiple non-invasive reporter usage within a single animal. BMC Biotechnol. 2, 11.
Kent, D. G., Copley, M. R., Benz, C., Wöhrer, S., Dykstra, B. J., Ma, E., Cheyne, J., Zhao, Y., Bowie, M. B., Zhao, Y., et al. (2009). Prospective isolation and molecular characterization of hematopoietic stem cells with durable self-renewal potential. Blood 113, 6342-6350.
Kiel, M. J., Yilmaz, O. H., Iwashita, T., Yilmaz, O. H., Terhorst, C., and Morrison, S. J. (2005). SLAM family receptors distinguish hematopoietic stem and progenitor cells and reveal endothelial niches for stem cells. Cell 121, 1109-1121.
Lane, S. W., Williams, D. a, and Watt, F. M. (2014). Modulating the stem cell niche for tissue regeneration. Nat. Biotechnol. 32, 795-803.
Lehrke, M. J. (2015). Dermatopontin Reduces Adhesion of Bone Marrow and Endothelial Cells in Vitro.
Ma, D., Zhang, J., Lin, H., Italiano, J., and Handin, R. (2011). The identification and characterization of zebrafish hematopoietic stem cells. Blood 118, 289-297.
Metcalf, D. (2008). Hematopoietic cytokines. Blood 111, 485-491.
Moore, K., Ema, H., and Lemischka, I. (1997a). In vitro maintenance of highly purified, transplantable hematopoietic stem cells. Blood 89, 4337-4347.
Moore, K., Ema, H., and Lemischka, I. R. (1997b). In vitro maintenance of highly purified, transplantable hematopoietic stem cells. Blood 89, 4337-4347.
Morrison, S. J., Shah, N. M., and Anderson, D. J. (1997). in Stem Cell Biology. Cell 88, 287-298.
Mosiewicz, K. A., Kolb, L., van der Vlies, A. J., Martino, M. M., Lienemann, P. S., Hubbell, J. A., Ehrbar, M., and Lutolf, M. P. (2013). In situ cell manipulation through enzymatic hydrogel photopatterning. Nat Mater 12, 1072-1078.
Muller-Sieburg, C. E., Cho, R. H., Karlsson, L., Huang, J.-F., and Sieburg, H. B. (2004). Myeloid-biased hematopoietic stem cells have extensive self-renewal capacity but generate diminished lymphoid progeny with impaired IL-7 responsiveness. Blood 103, 4111-4118.
Noda, S., Horiguchi, K., Ichikawa, H., and Miyoshi, H. (2008). Repopulating activity of ex vivo-expanded murine hematopoietic stem cells resides in the CD48-c-Kit+Sca-1+lineage marker-cell population. Stem Cells 26, 646-655.
Nolta, J. A., Thiemann, F. T., Dao, M. A., Barsky, L. W., Moore, K. A., Lemischka, I. R., and Crooks, G. M. (2002). The AFT024 stromal cell line supports long-term ex vivo maintenance of engrafting multipotent human hematopoietic progenitors. Leukemia 16, 352-361.
Notta, F., Doulatov, S., Laurenti, E., Poeppl, A., Jurisica, I., and Dick, J. E. (2011). Isolation of single human hematopoietic stem cells capable of long-term multilineage engraftment. Science 333, 218-221.
Okada, S., Nakauchi, H., Nagayoshi, K., Nishikawa, S., Miura, Y., and Suda, T. (1991). Enrichment and characterization of murine hematopoietic stem cells that express c-kit molecule. Blood 78, 1706-1712.
Oostendorp, R. a J., Harvey, K. N., Kusadasi, N., de Bruijn, M. F. T. R., Saris, C., Ploemacher, R. E., Medvinsky, A. L., and Dzierzak, E. a (2002). Stromal cell lines from mouse aorta-gonads-mesonephros subregions are potent supporters of hematopoietic stem cell activity. Blood 99, 1183-1189.
Osawa, M., Hanada, K., Hamada, H., and Nakauchi, H. (1996). Long-term lymphohematopoietic reconstitution by a single CD34-low/negative hematopoietic stem cell. Science (80-.). 273, 242-245.
Oswald, J., Steudel, C., Salchert, K., Joergensen, B., Thiede, C., Ehninger, G., Werner, C., and Bornhäuser, M. (2006). Gene-expression profiling of CD34+ hematopoietic cells expanded in a collagen I matrix. Stem Cells 24, 494-500.
Prewitz, M. C., Seib, F. P., von Bonin, M., Friedrichs, J., Stiflel, A., Niehage, C., Müller, K., Anastassiadis, K., Waskow, C., Hoflack, B., et al. (2013). Tightly anchored tissue-mimetic matrices as instructive stem cell microenvironments. Nat. Methods 10, 788-794.
Punzel, M., Gupta, P., and Verfaillie, C. M. (2002). The microenvironment of AFT024 cells maintains primitive human hematopoiesis by counteracting contact mediated inhibition of proliferation. Cell Commun Adhes. 9, 149-159.
Rieger, M., Hoppe, P. S., Smejkal, B. M., Eitelhuber, A. C., and Schroeder, T. (2009). Hematopoietic cytokines can instruct lineage choice. Science. 325, 217-218.
Sauvageau, G., Thorsteinsdottir, U., Eaves, C. J., Lawrence, H. J., Largman, C., Lansdorp, P. M., and Humphries, R. K. (1995). Overexpression of HOXB4 in hematopoietic cells causes the selective expansion of more primitive populations in vitro and in vivo. Genes Dev. 9, 1753-1765.
Sekulovic, S., Gasparetto, M., Lecault, V., Hoesli, C. a, Kent, D. G., Rosten, P., Wan, A., Brookes, C., Hansen, C. L., Piret, J. M., et al. (2011). Ontogeny stage-independent and high-level clonal expansion in vitro of mouse hematopoietic stem cells stimulated by an engineered NUP98-HOX fusion transcription factor. Blood 118, 4366-4376.
Sorrentino, B. (2004). Clinical strategies for expansion of haematopoietic stem cells. Nat Rev Immunol. 4, 878-888.
Spangrude, G. J., Heimfeld, S., and Weissman, I. L. (1988). Purification and characterization of mouse hematopoietic stem cells. Science (80-.). 241, 58-62.
Superti-Furga, A., Rocchi, M., Schafer, B., and Gitzelmann, R. (1993). Complementary DNA sequence and chromosomal mapping of a human proteoglycan-binding cell-adhesion protein (dermatopontin). Genomics 17, 463-467.
Takano, H., Ema, H., Sudo, K., and Nakauchi, H. (2004). Asymmetric division and lineage commitment at the level of hematopoietic stem cells: inference from differentiation in daughter cell and granddaughter cell pairs. J Exp Med. 199, 295-302.
Takeda, U., Utani, A., Wu, J., and Adachi, E. (2002). Targeted disruption of dermatopontin causes abnormal collagen fibrillogenesis. J Invest Dermatol. 119, 678-683.
Takizawa, H., Schanz, U., and Manz, M. G. (2011). Ex vivo expansion of hematopoietic stem cells: mission accomplished? Swiss Med. Wkly. 141, w13316.
Vanderperre, B., Lucier, J., Bissonnette, C., Motard, J., Tremblay, G., Vanderperre, S., Wisztorski, M., Salzet, M., Boisvert, F., and Roucou, X. (2013). Direct Detection of Alternative Open Reading Frames Translation Products in Human Significantly Expands the Proteome. PLoS One 8.
Wagers, A. J. (2012). The stem cell niche in regenerative medicine. Cell Stem Cell. 10(4):362-9.
Weissman, I. L. (2000). Translating Stem and Progenitor Cell Biology to the Clinic: Barriers and Opportunities. Science. 287, 1442-1446.
Wilson, A., and Trumpp, A. (2006). Bone-marrow haematopoietic-stem-cell niches. Nat Rev Immunol. 6, 93-106.
Wilson, A., Laurenti, E., and Oser, G. (2008). Hematopoietic stem cells reversibly switch from dormancy to self-renewal during homeostasis and repair. Cell 135, 1118-1129.
Wineman, J., Moore, K., Lemischka, I., and Müller-Sieburg, C. (1996). Functional heterogeneity of the hematopoietic microenvironment: rare stromal elements maintain long-term repopulating stem cells. Blood 87, 4082-4090.
Wohrer, S., Knapp, D. J. H. F., Copley, M. R., Benz, C., Kent, D. G., Rowe, K., Babovic, S., Mader, H., Oostendorp, R. A. J., and Eaves, C. J. (2014). Distinct Stromal Cell Factor Combinations Can Separately Control Hematopoietic Stem Cell Survival, Proliferation, and Self-Renewal. Cell Rep. 11, 1-12.
Zhang, C. C., Kaba, M., Ge, G., Xie, K., Tong, W., Hug, C., and Lodish, H. F. (2006). Angiopoietin-like proteins stimulate ex vivo expansion of hematopoietic stem cells. Nat. Med. 12, 240-245.

1. (canceled)

2. A method for maintaining hematopoietic stem and/or progenitor cells in culture, the method comprising culturing the hematopoietic stem and/or progenitor cells in the presence of dermatopontin (DPT) or a functional fragment thereof.

3. The method of claim 2, wherein DPT or a functional fragment thereof is added to the cell culture and/or wherein DPT or a functional fragment thereof is exogenously expressed by cells present in the culture.

4. The method of claim 3, wherein the amount of DPT or a functional fragment thereof added to the cell culture is at least 10 ng/ml.

5. The method of claim 3, wherein the cells that exogenously express DPT or a functional fragment thereof have been modified to carry an expression construct for the expression of DPT or a functional fragment thereof.

6. The method of claim 2, wherein the DPT or the functional fragment thereof is from the same species as the hematopoietic stem and/or progenitor cells to be cultured.

7. The method of claim 2, wherein the DPT is selected from human DPT as represented in SEQ ID NO: 1 or mouse DPT as represented in SEQ ID NO:4 or wherein the functional fragment of DPT is selected from the fragment of human DPT as represented in SEQ ID NO: 2 or the fragment of mouse DPT as represented in SEQ ID NO:5.

8. The method of claim 2, wherein the cell culture does not contain feeder cells.

9. The method of claim 2, wherein the cell culture does not contain cells other than the hematopoietic stem and/or progenitor cells to be cultured.

10. The method of claim 2, wherein the hematopoietic stem and/or progenitor cells are selected from human hematopoietic stem and/or progenitor cells obtained from bone marrow, umbilical cord blood and/or peripheral blood and/or from murine hematopoietic stem and/or progenitor cells obtained from bone marrow, yolk sac, AGM region, fetal liver, spleen and/or peripheral blood.

11. The method of claim 2, wherein the hematopoietic stem and/or progenitor cells are mammalian hematopoietic stem and/or progenitor cells.

12. The method of claim 2, wherein the hematopoietic stem and/or progenitor cells have not been engineered to express (an) exogenous protein(s) other than DPT or a functional fragment thereof.

13. The method of claim 2, wherein the hematopoietic stem and/or progenitor cells have not been engineered to over-express endogenously expressed proteins.

14. A cell culture medium for the maintenance of hematopoietic stem and/or progenitor cells, wherein the cell culture medium comprises a medium and dermatopontin (DPT) or a functional fragment thereof and further optionally comprises serum or serum replacement, one or more reducing agents, and/or one or more antibiotics.

15. A kit comprising dermatopontin (DPT) or a functional fragment thereof and at least one of:
(a) a cell culture medium;
(b) one or more cytokines;
(c) serum or serum replacement;
(d) one or more reducing agents; and
(e) one or more antibiotics.
